(19) United States
(12) Patent Application Publication
(10) Pub. No.: US 2026/0341414 A1
(43) Pub. Date: Oct. 29, 2026

(54) METHOD AND APPARATUS FOR TRAINING PREDICTION MODEL, ELECTRONIC DEVICE, COMPUTER-READABLE STORAGE MEDIUM, AND COMPUTER PROGRAM PRODUCT

(71) Applicant: Tencent Technology (Shenzhen) Company Limited, Shenzhen (CN)

(72) Inventors: Yu ZHAO, Shenzhen (CN); Yuxing LU, Shenzhen (CN); Jianhua YAO, Shenzhen (CN); Chenchen QIN, Shenzhen (CN); Bing HE, Shenzhen (CN)

(73) Assignee: Tencent Technology (Shenzhen) Company Limited, Shenzhen (CN)

(21) Appl. No.: 19/687,804

(22) Filed: May 26, 2026

Related U.S. Application Data
(63) Continuation of application No. PCT/CN2025/107551, filed on Jul. 8, 2025.

(30) Foreign Application Priority Data
Aug. 22, 2024 (CN) 202411162345.7

(51) Int. Cl. G16B 40/20; G06N 3/045; G06N 3/048; G06N 3/0895; G16B 20/20; G16B 30/10

(52) U.S. Cl. CPC G16B 40/20; G06N 3/045; G06N 3/048; G06N 3/0895; G16B 20/20; G16B 30/10

(57) ABSTRACT

This application relates to training a prediction model that includes: pre-training a first prediction model based on a plurality of first sample whole genome sequences to obtain a second prediction model; extracting a target dinucleotide site from a plurality of dinucleotide sites of each first sample whole genome sequence, and taking a sequence within a preconfigured window range including the target dinucleotide site as a second sample whole genome sequence; annotating an actual methylation status for the target dinucleotide site in each second sample whole genome sequence; and performing training processing on the second prediction model based on each annotated second sample whole genome sequence to obtain a trained third prediction model, the third prediction model being configured to predict a methylation status of a dinucleotide site in a to-be-detected whole genome sequence.

### RELATED APPLICATION

This application is a continuation of International Patent Application No. PCT/CN2025/107551, filed Jul. 8, 2025, which claims priority to Chinese Patent Application No. 2024111623457, filed on Aug. 22, 2024. The contents of International Patent Application No. PCT/CN2025/107551 and Chinese Patent Application No. 2024111623457 are incorporated herein by reference in their entirety.

### SEQUENCE LISTING

The instant application contains a Sequence Listing which has been submitted electronically in XML format via the USPTO patent electronic filing system and is hereby incorporated by reference in its entirety. Said XML copy, created on Jul. 10, 2026, is named “514935_PK347C1_Sequence_Listing.xml” and is 5,562 bytes in size.

### FIELD OF THE TECHNOLOGY

This application relates to an artificial intelligence technology, and in particular, to a method and apparatus for training a prediction model, an electronic device, a computer-readable medium, and a computer program product.

### BACKGROUND OF THE DISCLOSURE

Methylation refers to a process of catalytically transferring a methyl group from an active methyl compound to other compounds, and mainly involves adding a methyl group (—CH₃) to a deoxyribonucleic acid (DNA) molecule, which typically occurs at the 5^{th} carbon position of cytosine. An abnormal methylation status is closely related to various diseases, such as cancer, neurological diseases. By detecting the methylation status of dinucleotide sites (CpG dinucleotide sites) in DNA, it is possible to gain an in-depth understanding of gene expression regulation mechanisms and explore epigenetic etiologies of diseases.

Methylation status detection in the related art mainly relies on methylation-specific experiments, requiring complex experimental procedures and high sequencing costs. Meanwhile, due to the presence of noise and technical deviations, the related art may have a relatively high error rate when processing complex biological samples. In the related art, there is currently no effective way to improve an accuracy of methylation status prediction.

### SUMMARY

Embodiments of this application provide a method and apparatus for training a prediction model, an electronic device, a computer-readable storage medium, and a computer program product, which can improve an accuracy of predicting a methylation status of a gene site.

Technical solutions of the embodiments of this application are implemented in the following modes.

An embodiment of this application provides a method for training a prediction model, the method being applied to an electronic device, and the method including:
pre-training a first prediction model based on a plurality of first sample whole genome sequences to obtain a second prediction model;
extracting a target dinucleotide site from a plurality of dinucleotide sites of each first sample whole genome sequence, and taking a sequence within a preconfigured window range including the target dinucleotide site as a second sample whole genome sequence;
annotating an actual methylation status for the target dinucleotide site in each second sample whole genome sequence to obtain an annotated second sample whole genome sequence; and
performing training processing on the second prediction model based on each annotated second sample whole genome sequence to obtain a trained third prediction model, the third prediction model being configured to predict a methylation status of a dinucleotide site in a to-be-detected whole genome sequence.

An embodiment of this application provides an apparatus for training a prediction model, including:
a pre-training module, configured to pre-train a first prediction model based on a plurality of first sample whole genome sequences to obtain a second prediction model;
a sample acquisition module, configured to extract a target dinucleotide site from a plurality of dinucleotide sites of each first sample whole genome sequence, and taking a sequence within a preconfigured window range including the target dinucleotide site as a second sample whole genome sequence,
the sample acquisition module being configured to annotate an actual methylation status for the target dinucleotide site in each second sample whole genome sequence to obtain an annotated second sample whole genome sequence; and
a fine-tuning training module, configured to perform training processing on the second prediction model based on each annotated second sample whole genome sequence to obtain a trained third prediction model, the third prediction model being configured to predict a methylation status of a dinucleotide site in a to-be-detected whole genome sequence.

An embodiment of this application provides an electronic device, the electronic device including:
a memory, configured to store a computer-executable instruction; and
a processor, configured to, when executing the computer-executable instruction stored in the memory, implement the method for training a prediction model provided by the embodiment of this application.

An embodiment of this application provides a computer-readable storage medium, having a computer-executable instruction stored therein, the computer-executable instruction, when executed by a processor, implementing the method for training a prediction model provided by the embodiment of this application.

An embodiment of this application provides a computer program product, including a computer-executable instruction, the computer-executable instruction, when executed by a processor, implementing the method for training a prediction model provided by the embodiment of this application.

The embodiments of this application have the following beneficial effects.

Through pre-training, the prediction model is enabled to learn relevant features in whole genome sequences. In a training phase, whole genome sequences within a specific range are configured to perform targeted training, thereby improving an accuracy of the prediction model in predicting the methylation status, by training a prediction model for the methylation status. By limiting a range of training data, data complexity is reduced, training efficiency is improved, and computational resources required for a training process are saved. By detecting a to-be-detected genome sequence using a trained prediction model, compared with methylation status detection in the related art, there is no need to perform complex experiments, noise and interference introduced during an experimental process are avoided, and an efficiency and an accuracy of the methylation status detection are improved.

### BRIEF DESCRIPTION OF THE DRAWINGS

FIG. 1 is a schematic diagram of an application mode of a method for training a prediction model provided by an embodiment of this application.

FIG. 2 is a schematic structural diagram of an electronic device provided by an embodiment of this application.

FIG. 3A is a first schematic flowchart of a method for training a prediction model provided by an embodiment of this application.

FIG. 3B is a second schematic flowchart of a method for training a prediction model provided by an embodiment of this application.

FIG. 3C is a third schematic flowchart of a method for training a prediction model provided by an embodiment of this application.

FIG. 3D is a fourth schematic flowchart of a method for training a prediction model provided by an embodiment of this application.

FIG. 3E is a fifth schematic flowchart of a method for training a prediction model provided by an embodiment of this application.

FIG. 4 is a first schematic structural diagram of a prediction model provided by an embodiment of this application.

FIG. 5 is a sixth schematic flowchart of a method for training a prediction model provided by an embodiment of this application.

FIG. 6A is a first schematic diagram of a training principle provided by an embodiment of this application.

FIG. 6B is a second schematic diagram of a training principle provided by an embodiment of this application.

FIG. 6C is a second schematic structural diagram of a prediction model provided by an embodiment of this application.

FIG. 6D is a schematic diagram of a data flow of a prediction model provided by an embodiment of this application.

The terms “first”, “second”, and so on, described above are only configured to distinguish between different solutions, and are not intended to indicate a degree of superiority or a priority in implementation.

### DESCRIPTION OF EMBODIMENTS

In order to make the objectives, technical solutions, and advantages of this application clearer, this application is further described in detail below with reference to the accompanying drawings. The described embodiments are not to be construed as limiting this application. All other embodiments obtained by a person skilled in the art without making creative efforts fall within the scope of protection of this application.

In the following descriptions, “some embodiments” involved describe a subset of all possible embodiments. However, “some embodiments” may be a same subset or different subsets of all possible embodiments, and may be combined with each other without conflict.

In the following description, the terms “first”, “second”, “third” involved are only configured to distinguish between similar objects and do not denote a specific order of the objects. Where permitted, the specific order or sequence indicated by “first”, “second”, “third” may be interchanged, so that the embodiments of this application described herein may be implemented in an order other than that illustrated or described herein.

In this application, when a biometric feature (e.g., a DNA sequence or a biological sample) recognition technology involved is applied to a specific product or technology in the above embodiments of this application, the collection, use, and processing of relevant data are to comply with the requirements of relevant laws and regulations. Before collecting biometric features, information processing rules are to be communicated and separate consent from a target subject is to be obtained. Moreover, the biometric features are to be processed in strict accordance with the requirements of laws and regulations and personal information processing rules, and technical measures are to be taken to ensure a security of relevant data.

In the embodiment of this application, the term “module” or “unit” refers to a computer program with a preset function or a part of the computer program and works, together with other related parts, to implement a preset target, and may be completely or partially implemented by using software, hardware (for example, a processing circuit or a memory) or a combination thereof. Similarly, one processor (or a plurality of processor or memories) may be configured to realize one or more modules or units. In addition, each module or unit may be a part of an overall module or unit including the module or unit function.

Unless otherwise defined, all technical and scientific terms used herein have a same meaning as commonly understood by a person skilled in the art of this application. The terms used herein are for the purpose of describing embodiments of this application only and are not intended to limit this application.

Before further detailed description of the embodiments of this application, the nouns and terms involved in the embodiments of this application are explained, and the nouns and terms involved in the embodiments of this application are subject to the following explanations.
1) Deoxyribonucleic acid (DNA): It is an important molecule in cells that carries genetic information of an organism, hereinafter referred to as DNA. DNA includes two long strands that are intertwined in a form of a double helix. Each long strand includes many small nucleotide units, and each nucleotide unit includes a phosphate group, a sugar (deoxyribose), and a nitrogenous base. DNA has four types of nitrogenous bases: adenine (A), thymine (T), cytosine (C), and guanine (G). At corresponding positions on the two long strands, adenine always pairs with thymine, and cytosine always pairs with guanine. These base pairs are connected to each other by hydrogen bonds, forming the double helix structure of DNA.
2) Cytosine: The scientific name is 4-amino-2-carbonylpyrimidine, and it is an organic compound with a molecular formula C₄H₅N₃O. It is one of the main base components of nucleic acids (DNA and RNA).
3) CpG dinucleotide sites: These are a special type of DNA sequence, also referred to as CpG sites or CG sites for short, in which the base sequence is cytosine (C) and guanine (G), linked by a phosphodiester bond.
4) Methyl group: a chemical group with the chemical formula —CH₃ (“—” represents a single electron), abbreviated as -Me, including carbon and hydrogen elements. It is a monovalent, electrically neutral group remaining after removing a hydrogen atom from a methane molecule. As a chemical group, the methyl group often appears in various organic compounds and is the most common group. It can bind to certain specific sites on DNA. The process of a methyl group binding to DNA is called methylation, and conversely, the process of detaching from a compound is called hypomethylation.
5) Methylation: This refers to the process of catalytically transferring a methyl group from an active methyl compound to other compounds. The methylation process can form various methyl compounds, or chemically modify certain proteins or nucleic acids to form methylated products. In biological systems, methylation is catalyzed by enzymes, and such methylation involves heavy metal modification, regulation of gene expression, regulation of protein function, and RNA processing. DNA methylation is a form of chemical modification of DNA that can alter genetic expression without changing the DNA sequence. So-called DNA methylation refers to the covalent bonding of a methyl group at the 5^{th} carbon position of cytosine in a genomic dinucleotide (CpG) under the action of DNA methyltransferase. Methylation status includes various types: 1. Unmethylated: no methyl group (—CH₃) is added at the 5^{th} carbon position of cytosine (C), i.e., the original CG site. 2. Partially methylated: a methyl group (—CH₃) is partially added at the 5^{th} carbon position of cytosine (C), i.e., part of the CG site is converted to 5-methylcytosine (mC). 3. Completely methylated: a methyl group (—CH₃) is completely added at the 5^{th} carbon position of cytosine (C), i.e., the CG site is completely converted to 5-methylcytosine (mC). 4. Hypermethylation: an excessive amount of methyl groups (—CH₃) is added at the 5^{th} carbon position of cytosine (C), causing the methylation level of the CG site to exceed a normal level. 5. Hypomethylation: the methyl group (—CH₃) at the 5^{th} carbon position of cytosine (C) is reduced or completely removed, causing the methylation level of the CG site to fall below a normal level.
6) Whole genome sequencing (WGS): A high-throughput sequencing technology configured to acquire and study the entire genetic information of an organism, hereinafter referred to as WGS. Through WGS, the base sequences of all genes, non-coding sequences, and mitochondrial DNA of an organism can be determined.
7) Whole-genome bisulfite sequencing (WGBS): A sequencing technology configured to detect DNA methylation status across the entire genome. Bisulfite treatment is configured to convert unmethylated cytosine (C) to uracil (U), while methylated cytosine remains unchanged. After treatment, the DNA is amplified by polymerase chain reaction (PCR) and sequenced. Through bioinformatics analysis of the original sequencing data, the methylation status of individual C bases across the entire genome can be reconstructed.
8) Transformer model: The model is a deep neural network model based on a self-attention mechanism, widely used in various tasks in the field of natural language processing, such as text classification, machine translation, and question answering systems. The model can convert an input sequence into an output sequence while retaining important information in the input sequence. Due to its excellent performance in processing long texts, the transformer model has been widely applied in the field of Chinese natural language processing. Compared with conventional recurrent neural networks (RNNs) and convolutional neural networks (CNNs), the transformer model allows parallel computation, accelerating a training speed. It has been widely applied in tasks such as natural language processing, speech recognition, and image generation.

In the related art, methylation is an epigenetic modification phenomenon that mainly involves adding a methyl group (—CH₃) to a DNA molecule, typically occurring at the 5^{th} carbon position of cytosine. An abnormal methylation status is closely related to various diseases, such as cancer, neurological diseases. In the related art, the methylation status is determined by whole-genome bisulfite sequencing, but this experimental operation involves complex data processing. Due to the presence of noise and technical deviations, whole-genome bisulfite sequencing may have a relatively high error rate when processing complex biological samples.

Embodiments of this application provide a method for training a prediction model, an apparatus for training a prediction model, an electronic device, a computer-readable storage medium, and a computer program product, which can improve an accuracy of predicting a methylation status of a gene site.

The following describes exemplary applications of the electronic device provided by the embodiment of this application. The electronic device provided by the embodiment of this application may be implemented as a terminal device, such as a laptop computer, a tablet computer, a desktop computer, a set-top box, a smart television, a vehicle-mounted terminal, a virtual reality (VR) device, an augmented reality (AR) device, or other types of terminals, or may be implemented as a server. Hereinafter, exemplary applications when the electronic device is implemented as a terminal device or a server will be described.

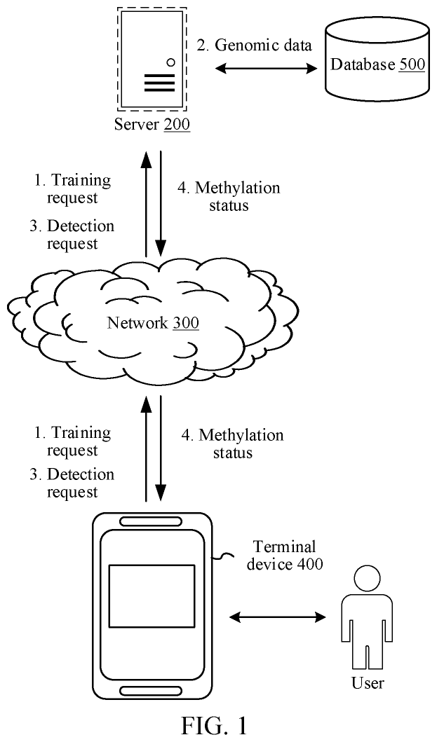

Referring to FIG. 1, FIG. 1 is a schematic diagram of an application mode of a method for training a prediction model provided by an embodiment of this application. Exemplarily, FIG. 1 shows a server 200, a network 300, a terminal device 400, and a database 500. The terminal device 400 is connected to the server 200 via the network 300. The network 300 may be a wide area network, a local area network, or a combination thereof.

Exemplarily, a user may be a technician, the terminal device 400 may be a computer used by the technician, the server 200 may be a server of a data processing platform, and the database 500 stores a large amount of genomic data.

Exemplarily, the user transmits a training request to the server 200 via the terminal device 400. The server 200 extracts, based on the training request, genomic data corresponding to the training request from the database 500. The server 200 calls the method for training a prediction model provided by the embodiment of this application to train a prediction model to obtain a trained prediction model, and the trained prediction model is stored in the server 200. When the user transmits to-be-detected genomic data via the terminal device 400, or the terminal device 400 transmits a detection request to the server 200, the server 200 extracts, based on a data identifier carried in the detection request, the to-be-detected genomic data from the database 500. The server 200 performs prediction processing on the to-be-detected genomic data using a trained prediction model, obtains a methylation status of a dinucleotide site in the to-be-detected genomic data, and transmits data related to the methylation status to the terminal device 400. The technician may perform relevant analysis based on the methylation status of the to-be-detected genomic data.

In some embodiments, the method for training a prediction model provided by the embodiment of this application may also be applied in the following application scenarios:
1. Disease diagnosis: Predicting the methylation status of gene sites related to diseases can help scientists understand the pathogenesis of diseases, identify biomarkers for diseases, and develop new treatment methods. A prediction model is trained by the method for training a prediction model provided by the embodiment of this application. The prediction model is configured to perform prediction processing on a DNA sequence in a blood sample of a patient to obtain a methylation status of a dinucleotide site in the DNA sequence. Researchers can diagnose whether the patient has a related disease based on the position of the dinucleotide site in the DNA sequence and the methylation status of the dinucleotide site.

As an example, in colorectal cancer diagnosis, researchers use a trained third prediction model to perform prediction processing on a fecal DNA sample of a patient. The model input is a whole genome sequence (extracting a target dinucleotide site, such as a CpG island in the SEPT9 gene region), and the output is the methylation status of these sites. If the model predicts that the proportion of hypermethylation at key sites exceeds a threshold (e.g., >80%), a high risk of cancer is indicated, and a doctor may recommend that the patient undergo a colonoscopy accordingly. This method is more sensitive than conventional methods and can reduce a false negative rate, and has been applied in clinical research (reference source: Guidelines for Epigenetic Screening, such as studies related to liquid biopsy).
2. Drug development: during the drug development process, predicting the methylation status of a drug target can help optimize drug design and improve drug efficacy. A prediction model is trained by the method for training a prediction model provided by the embodiment of this application. The prediction model is configured to perform prediction processing on data of a biological macromolecule to obtain a methylation status of a target on the biological macromolecule that is specifically acted upon by a drug, a therapeutic protein, or other intervention measures. Then, based on the methylation status of the target, researchers can correspondingly design drug molecules that readily bind to the target.

As an example, in leukemia treatment, researchers train a third prediction model based on annotated second sample whole genome sequences (from a patient's bone marrow cells). Then, the model is applied to predict on whole genome data of a patient in a clinical trial: the input is sequences before/after drug treatment (a preconfigured window covering the DNMT3A gene site), and the output is a change in the methylation status of a target dinucleotide site. If the model predicts a significant decrease in the methylation level at a key site (e.g., positively correlated with drug response), researchers can screen out a high-response patient population accordingly and optimize the drug dosage (e.g., using Azacitidine). This reduces trial-and-error costs and increases the treatment success rate (reference source: Application of epigenetics in cancer drug development, such as Nature Reviews Drug Discovery).
3. Agricultural breeding and crop improvement: In agricultural biotechnology, the model can be configured to predict the methylation status of dinucleotide sites in the whole genome of a crop or livestock to associate with environmental response traits (e.g., drought resistance and disease resistance). The methylation status can reflect epigenetic variation, affecting gene expression without altering the DNA sequence itself. The present solution enhances a prediction accuracy of the model for specific trait sites through local window training, helping breeders quickly screen superior individuals and shorten the breeding cycle.

As an example: in rice breeding, researchers use a third prediction model to perform prediction processing on whole genome sequences of a large number of rice strains. The model input is an extracted target dinucleotide site (e.g., a window sequence of the OsWRKY gene related to resistance to rice blast), and the output is the methylation status. Based on the prediction results (e.g., hypomethylation at certain sites being positively correlated with disease resistance), breeders can preferentially select rice lines with these methylation patterns for crossbreeding experiments. This is more efficient than conventional genetic editing and can increase yield under drought or disease conditions (reference source: Research on crop epigenetic breeding, such as Plant Biotechnology Journal).

In some embodiments, the server 200 may be an independent physical server, or may be a server cluster or a distributed system composed of a plurality of physical servers. For example, the server 200 may be a server cluster or a distributed system composed of a training server and a data prediction server. The training server is configured to train a prediction model, and when a trained prediction model is obtained, the training server transmits the trained prediction model to the data prediction server. The data prediction server is configured to perform, based on the stored prediction model, prediction processing on a to-be-detected gene sequence to obtain a methylation status of a dinucleotide site in the gene sequence.

In some embodiments, the server 200 may be an independent physical server, or may be a server cluster or a distributed system composed of a plurality of physical servers. The terminal and the server may be connected directly or indirectly through wired or wireless communication, which is not limited in the embodiment of this application.

The embodiment of this application may be implemented through database technology. A database, in simple terms, can be regarded as an electronic file cabinet, i.e., a place for storing electronic files, and a user may perform operations such as adding, querying, updating, and deleting data in the files. A so-called “database” is a data collection that is stored together in a certain mode, can be shared by a plurality of users, has as little redundancy as possible, and is independent of application programs.

A database management system (DBMS) is a computer software system designed for managing databases, generally having basic functions such as storage, retrieval, security assurance, and backup. The database management system may be classified according to the database model it supports, for example, relational, XML (Extensible Markup Language); or according to the type of computer it supports, for example, server clusters, mobile phones; or according to the query language it uses, for example, Structured Query Language (SQL), XQuery; or according to the performance metrics it focuses on, for example, maximum scale, highest running speed; or other classification methods. Regardless of which classification method is used, some DBMSs can cross categories, for example, supporting multiple query languages at the same time.

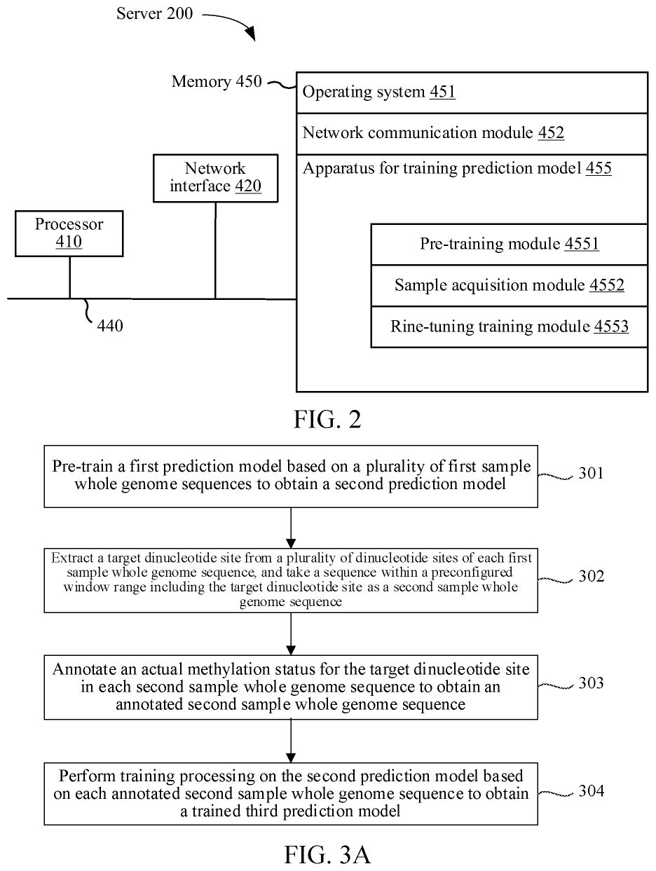

Referring to FIG. 2, FIG. 2 is a schematic structural diagram of an electronic device provided by an embodiment of this application. The electronic device may be the server 200 shown in FIG. 1. The server 200 shown in FIG. 2 includes: at least one processor 410, a memory 450, and at least one network interface 420. Various components in the server 200 are coupled together by using a bus system 440. The bus system 440 is configured to implement connection and communication between these components. In addition to a data bus, the bus system 440 further includes a power bus, a control bus, and a state signal bus. However, for ease of clear description, all types of buses are marked as the bus system 440 in FIG. 2.

The processor 410 may be an integrated circuit chip with signal processing capacity such as a general-purpose processor, a digital signal processor (DSP), another programmable logic device, discrete gate or transistor logic device, or discrete hardware assembly. The general-purpose processor may be a microprocessor, any conventional processor, or the like.

The memory 450 may be a removable memory, a non-removable memory, or a combination thereof. An exemplary hardware device includes a solid memory, a hard disk drive, an optical disk drive, and the like. In some embodiments, the memory 450 includes one or more storage devices away from the processor 410 in a physical position.

The memory 450 includes a volatile memory or a non-volatile memory, or may include both a volatile memory and a non-volatile memory. The non-volatile memory may be a read-only memory (ROM). The volatile memory may be a random access memory (RAM). The memory 450 described in the embodiment of this application is intended to include any suitable type of memory.

In some embodiments, the memory 450 can store data to support various operations. An example of these data includes a program, a module, and a data structure or a subset or superset thereof, which will be exemplarily described below.

An operating system 451 includes system programs for processing various basic system services and executing hardware-related tasks, such as a framework layer, a core library layer, and a drive layer, and is configured to implement various basic services and process hardware-based tasks.

A network communication module 452 is configured to reach another electronic device via one or more (wired or wireless) network interfaces 420. Exemplary network interfaces 420 include Bluetooth, wireless fidelity (Wi-Fi), a universal serial bus (USB), and the like.

In some embodiments, the apparatus provided by the embodiment of this application may be implemented in software. FIG. 2 shows an apparatus 455 for training a prediction model stored in the memory 450, which may be software in the form of a program, a plug-in, or the like, including the following software modules: a pre-training module 4551, a sample acquisition module 4552, and a fine-tuning training module 4553. These modules are logical, and thus may be combined in different modes to form other embodiments or further split according to the functions implemented. The functions of each module will be described below.

In some embodiments, the terminal device or the server may implement the method for training a prediction model provided by the embodiment of this application by executing a computer program. For example, the computer-executable instruction may be microprogram-level commands, machine instructions, or software instructions. The computer program may be a native program or a software module in an operating system; may be a native application (APP), i.e., a program that needs to be installed in an operating system to run, such as an instant messaging APP; or may be a small program that can be embedded into any APP, i.e., a program that only needs to be downloaded into a browser environment to run. In summary, the computer-executable instruction described above may be in any form of instructions, and the computer program described above may be in any form of application program, module, or plug-in.

The method for training a prediction model provided by the embodiment of this application will be described in conjunction with exemplary applications and implementations of the server or the terminal device provided by the embodiment of this application.

Hereinafter, the method for training a prediction model provided by the embodiment of this application will be described. As described above, the electronic device implementing the method for training a prediction model provided by the embodiment of this application may be a terminal device or a server, or a combination thereof. Therefore, the execution subject of each operation will not be repeatedly described below.

In the following examples of methylation status prediction processing, whole genome sequence data is used as an example for description. Based on the understanding of the following description, a person skilled in the art may apply the method for training a prediction model provided by the embodiment of this application to processing other types of genetic data, such as RNA sequencing data (RNA-seq) and chromatin immunoprecipitation sequencing data (ChIP-seq).

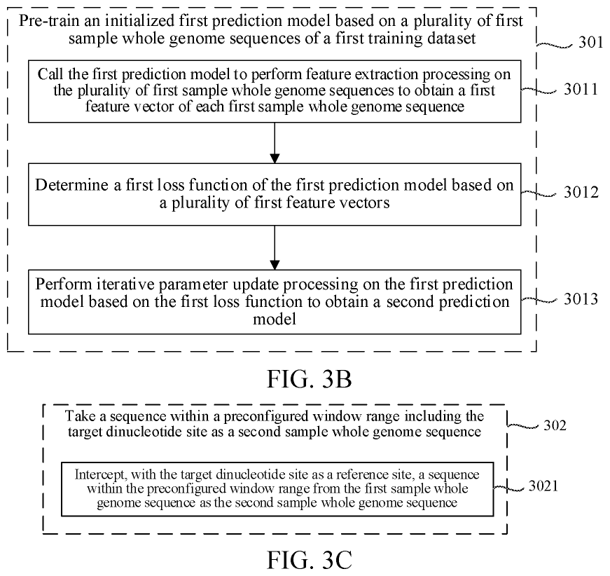

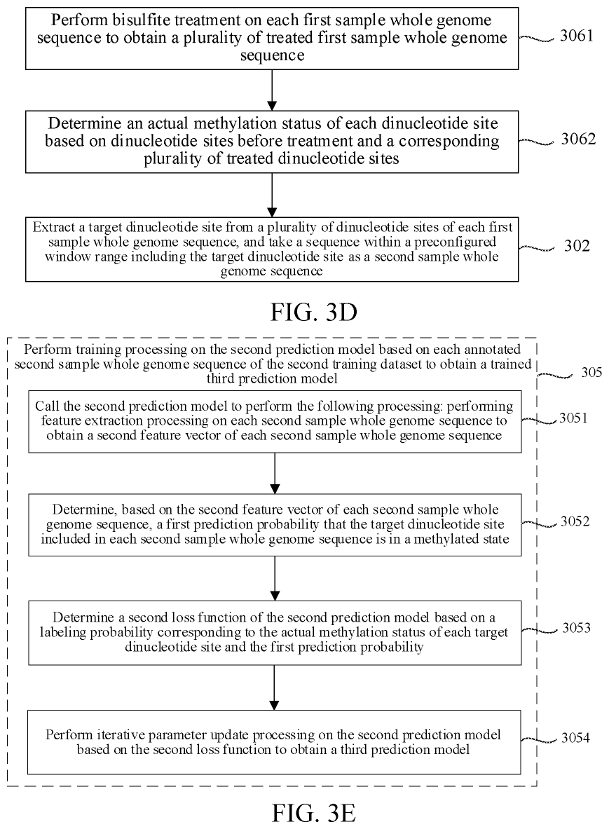

Referring to FIG. 3A, FIG. 3A is a schematic flowchart of a method for training a prediction model provided by an embodiment of this application. The description will be made in conjunction with the operations shown in FIG. 3A. The execution subject of the operations in FIG. 3A is the server 200 in FIG. 1.

To facilitate understanding, an application scenario of the method for training a prediction model according to the embodiment of this application will be explained. A methyl group is a chemical group that can bind to specific sites on DNA. Methylation refers to a process of catalytically transferring a methyl group from an active methyl compound to other compounds. Methylation status includes unmethylated, partially methylated, completely methylated, hypermethylated, and hypomethylated states. A dinucleotide site (CG site) includes cytosine (C) and guanine (G) and is a component of deoxyribonucleic acid (DNA). DNA methylation refers to the covalent bonding of a methyl group at the 5^{th} carbon position of cytosine in a genomic dinucleotide (CpG) under the action of DNA methyltransferase. In the embodiment of this application, the methylation status of a dinucleotide site is classified into unmethylated and methylated states, where a methylated state includes partially methylated, completely methylated, hypermethylated, and hypomethylated states. Based on the above scenario, the method for training a prediction model provided by the embodiment of this application will be explained and described below.

Operation 301: Pre-train a first prediction model based on a plurality of first sample whole genome sequences to obtain a second prediction model.

Exemplarily, the first prediction model includes an embedding layer, a transformer block, and a prediction head. The first prediction model and the second prediction model, and the third prediction model described below have a same structure, with “first”, “second”, and “third” being configured to distinguish different stages of the same model. A whole genome sequence is the entire genetic information of an organism obtained by performing whole genome sequencing (WGS) processing on DNA. A source of DNA may be a biological sample, such as a blood sample, and a hair sample.

Exemplarily, the pre-training processing is configured to train a function of the first prediction model to perform feature extraction on the first sample whole genome sequences, so as to obtain the second prediction model. A mode of pre-training processing may be self-supervised training. Self-supervised training is a machine learning training method that does not rely on externally annotated data for learning. In self-supervised training, a model obtains knowledge by learning the intrinsic structure and patterns of input data, automatically generating supervisory signals.

In some embodiments, before operation 301, real whole genome sequences and a reference whole genome sequence are acquired to construct a first training dataset. The first training dataset may be acquired in the following mode: performing whole genome sequencing processing on a biological sample to obtain a plurality of real whole genome sequences; and taking a preconfigured reference whole genome sequence and the plurality of real whole genome sequences as first sample whole genome sequences.

The embodiment of this application breaks the limitation of a single reference and construct a dynamic reference benchmark that is closer to the genetic diversity of a study population, thereby improving the accuracy of pre-training of the first prediction model.

Exemplarily, the biological sample is, for example, a blood sample collected in cancer screening. In practical application scenarios, the biological sample is collected under the conditions that the subject from whom the biological sample is collected has given consent and the source is legal. The reference whole genome sequence may be an artificially edited whole genome sequence or a whole genome sequence that has already been sequenced, and the number of reference whole genome sequences is at least one.

To facilitate understanding, an example of a whole genome sequence is provided below: “ . . . . AACAACGGTAGATCAGGATTAGA . . . ” (SEQ ID NO:1). DNA includes two long strands that are intertwined in the form of a double helix. DNA has four types of nitrogenous bases: adenine (A), thymine (T), cytosine (C), and guanine (G). At corresponding positions on the two long strands, adenine always pairs with thymine, and cytosine always pairs with guanine. Therefore, by determining the base sequence of one long strand of DNA, the base sequence of the other long strand of DNA can be determined.

In some embodiments, the performing whole genome sequencing processing on a biological sample to obtain a plurality of real whole genome sequences may be implemented by the following technical solution: fragmenting deoxyribonucleic acid, and preparing a fragmentation result into text data for sequencing; sequencing the text data to obtain a plurality of fragment sequencing reads; determining an arrangement order of bases in a genome sequence based on the plurality of fragment sequencing reads; and reconstructing the genome sequence based on the arrangement order to obtain the plurality of real whole genome sequences.

Whole genome sequencing processing may be implemented in the following mode: fragmenting extracted DNA using a specific method and preparing fragmented DNA into text data for sequencing; sequencing constructed text data using a high-throughput sequencing platform (e.g., Illumina or SOLID) to obtain a large number of short fragment sequencing reads; and processing, aligning, performing variation detection and annotation on an original data obtained by sequencing to obtain bases in the genome sequence and the arrangement order of the bases, so as to reconstruct the entire genome sequence. The above process typically involves a complex bioinformatics analysis process, including quality control, mapping, assembly, variation detection, functional annotation, and the like.

As an example, long-chain DNA is randomly fragmented into small fragments suitable for a read length of a sequencing platform. The fragmented DNA is processed into a format recognizable by a sequencer (e.g., adapter addition, amplification, purification, fragment selection). The sequences at both ends of library fragments are read on the sequencer. A large number of unordered short reads, which may contain errors, are mapped back to a reference genome or assembled into contiguous sequences, and the base at each position is determined. A complete, individual-specific whole genome sequence for each sample is obtained from the alignment or assembly results.

The embodiment of this application combines mature experimental operations and powerful bioinformatics analysis processes, thereby enabling the acquisition of individualized, high-quality complete genome sequences.

In some embodiments, a self-supervised training mode is adopted. Referring to FIG. 3B, FIG. 3B is a second schematic flowchart of a method for training a prediction model provided by an embodiment of this application. Operation 301 may be implemented through operations 3011 to 3013 in FIG. 3B, which will be described in detail below.

Operation 3011: Call the first prediction model to perform feature extraction processing on the plurality of first sample whole genome sequences to obtain a first feature vector of each first sample whole genome sequence.

Exemplarily, the first prediction model includes an embedding layer, and the embedding layer is configured to map each first sample whole genome sequence into a feature vector in the form of a token embedding vector. For example, data corresponding to a base at each site in the first sample whole genome sequence is mapped into a token representation, and the token representation corresponding to each site is sequentially combined to obtain the first feature vector.

A purpose of operation 3011 is to extract meaningful, low-dimensional numerical representations (i.e., first feature vectors) from the original genomic data using an existing first prediction model. The goal of this operation is to convert an original, high-dimensional, complex whole genome sequence (a long string including A, T, C, G bases) into a fixed-length numerical vector (a first feature vector) containing rich information. This provides a more easily processable and informative input for the subsequent construction of the second prediction model.

The input is a plurality of first sample whole genome sequences, which are the original input data, and each sample represents a complete genome sequence (e.g., DNA from different individuals or cells).

The first prediction model is a model which has already been trained. It is typically a deep neural network (e.g., CNN, Transformer, or variants thereof), whose core capability lies in understanding the contextual information, patterns (e.g., motifs), and potential structures of DNA sequences. The model is trained on the first training dataset, meaning that it has learned to extract useful, general features from raw DNA sequences.

Internally, when a whole genome sequence is input into the model, the model processes it layer by layer: The bottom layers may recognize local patterns (e.g., specific k-mer combinations), the middle layers may combine these local patterns to identify more complex structures or contextual relationships, and before the final layers (or a specifically designed output layer), the model internally forms a high-dimensional, abstract internal representation of the input sequence. This internal representation (typically the activation values of a certain layer near the end of the model or of a specific pooling layer) is extracted as the “first feature vector” for the sample, and the first feature vector is finally output.

For each input first sample whole genome sequence, after being processed by the first prediction model, a corresponding fixed-length numerical vector is output. This vector is no longer the original base sequence, but a compressed, abstract representation of the overall or key characteristics of the sequence learned by the model. It contains information that the model considers useful for understanding the sequence structure and potential biological significance (even if methylation is not directly involved at this point).

Through operation 3011, a corresponding fixed-dimensional feature vector (first feature vector) is generated for each input whole genome sequence. These vectors are more compact and information-dense than the original sequences, providing a high-quality input foundation for the subsequent construction of a second prediction model that focuses more on specific tasks (e.g., predicting methylation). In essence, this is a key preprocessing operation that converts high-dimensional sequence data into numerical representations that are easier for a model to learn.

Operation 3012: Determine a first loss function of the first prediction model based on a plurality of first feature vectors.

Exemplarily, in self-supervised training, the first loss function is a function configured to measure similarities between feature vectors of samples with the same content and similarities between feature vectors of samples with different contents. In the self-supervised training process, the goal is to increase the similarity between samples with the same content and decrease the similarity between samples with different contents.

In some embodiments, operation 3012 may be implemented in the following mode: performing, for each first sample whole genome sequence, the following processing: determining a first similarity between the first feature vector of the first sample whole genome sequence and a first feature vector of a third sample whole genome sequence, and determining a second similarity between the first feature vector of the first sample whole genome sequence and a first feature vector of a fourth sample whole genome sequence, where the third sample whole genome sequence is another first sample whole genome sequence having a same base sequence as the first sample whole genome sequence, and the fourth sample whole genome sequence is another first sample whole genome sequence having a different base sequence from the first sample whole genome sequence; taking a first ratio of the first similarity to a first sum as a first sub-loss function corresponding to the first sample whole genome sequence, where the first sum is a sum of the first similarity and the second similarity; and taking a mean of a plurality of first sub-loss functions as the first loss function of the first prediction model.

Exemplarily, assuming that a first sample whole genome sequence i is any one of all first sample whole genome sequences, a first sample whole genome sequence j having the same base sequence as the first sample whole genome sequence i is acquired, and a first sample whole genome sequence 1 having a different base sequence from the first sample whole genome sequence i is acquired. The first similarity and the second similarity are acquired respectively, and the first similarity and the second similarity may be cosine similarities between feature vectors. The first ratio may be represented by the following formula: first similarity/(first similarity+second similarity).

Exemplarily, the loss function used in self-supervised training may specifically be contrastive loss or information regularization loss. Contrastive loss is a loss function used in self-supervised learning. It aims to learn a model that can distinguish positive sample pairs (i.e., related samples) from each other while bringing negative sample pairs (i.e., unrelated samples) closer together. The application of contrastive loss in NLP is typically for learning embedded representations of text, which can be configured for subsequent tasks such as text classification, information retrieval.

Information regularization loss is a loss function widely used in self-supervised learning, particularly in the setting of contrastive learning. The purpose of information regularization loss is to enable the model to learn to distinguish between positive sample pairs (similar samples) and negative sample pairs (dissimilar samples), while penalizing overly high similarity judgments for positive sample pairs to avoid overfitting.

Through the embodiment of this application, by comparing the similarity of samples with the same/different sequences, the feature vector is forced to retain genetic information sensitive to base arrangement patterns (e.g., haplotypes, structural variations), and to filter out noise unrelated to genetic differences (e.g., sequencing errors, batch effects), thereby optimizing a training effect of supervised training as a whole.

Operation 3013: Perform iterative parameter update processing on the first prediction model based on the first loss function to obtain the second prediction model.

Exemplarily, the second prediction model is configured to extract a feature vector of a to-be-detected whole genome sequence. The number of iterations and the learning rate used in the parameter update processing may be set according to actual application scenarios. The mode of parameter update processing is backpropagation processing.

In the embodiment of this application, the pre-training processing is implemented by way of self-supervised training. The pre-training process can be completed without requiring manual or additional machine annotation of sample data. Since the data used in self-supervised training is unannotated, the model needs to learn to automatically extract useful feature representations from the data, which helps reduce the risk of overfitting. Enabling the prediction model to learn richer feature representations helps improve the generalization capability of the prediction model.

In some embodiments, the first sample whole genome sequence includes a plurality of dinucleotide sites. Referring to FIG. 3D, FIG. 3D is a fourth schematic flowchart of a method for training a prediction model provided by an embodiment of this application. Before operation 302, the actual methylation status of each dinucleotide site may be determined through operations 3061 to 3062 in FIG. 3D, as described in detail below.

Operation 3061: Perform bisulfite treatment on each first sample whole genome sequence to obtain a plurality of treated first sample whole genome sequences.

Here, each treated first sample whole genome sequence includes a plurality of treated dinucleotide sites.

Exemplarily, whole-genome bisulfite sequencing is a method for determining DNA methylation status. Its principle is to convert unmethylated cytosine (C) to uracil (U) through bisulfite treatment. After bisulfite treatment, a dinucleotide site in a methylated state in the original DNA will appear as GpG, while an unmethylated dinucleotide site will appear as CpG or UpG.

Bisulfite treatment is a core experimental technique for detecting DNA methylation status. This operation provides basic data for constructing a methylation “ground truth label” for the first sample whole genome sequences in the technical solution. The purpose of operation 3061 is to distinguish between methylated and unmethylated cytosine (C). Bisulfite treatment converts unmethylated cytosine (C) in DNA to uracil (U), while methylated cytosine (5mC) remains unchanged. In subsequent PCR amplification or sequencing, uracil (U) is recognized as thymine (T). Therefore, the original sequence C→sequencing showing T indicates that the site is unmethylated; and the original sequence C→sequencing still showing C indicates that the site is methylated.

The operations of bisulfite treatment are as follows: DNA denaturation: double-stranded DNA samples are denatured into single strands under alkaline conditions to ensure that bisulfite can fully contact cytosine; bisulfite treatment: single-stranded DNA reacts with bisulfite (e.g., sodium bisulfite): unmethylated C→conversion to sulfonated cytosine→deamination under alkaline conditions→generation of uracil (U), while methylated C (5mC)→extremely slow reaction rate→remains substantially unchanged; purification and desulfonation: removal of residual chemical reagents and conversion of sulfonated intermediates into stable uracil (U); PCR amplification and sequencing: DNA is amplified using a polymerase that has no bias toward uracil (U), and the converted sequence is finally obtained by sequencing.

Original sequence . . . ACG . . . (assuming that the second C is unmethylated)→becomes . . . ATG . . . after treatment (i.e., C→T conversion, while methylated C remains C).

Generating training labels: by comparing the sequence differences before and after treatment, the actual methylation status of each cytosine site (especially CpG dinucleotides) can be directly annotated. These annotated data are used in the pre-training stage, for constructing feature vectors of the first prediction model (e.g., self-supervised learning), and in the fine-tuning training stage, for providing supervisory signals for the second prediction model (e.g., annotating the methylation status of target CpG sites). The treated sequences constitute “gold standard” data for subsequent model training and validation, ensuring that the model prediction results have biological interpretability.

Operation 3062: Determine an actual methylation status of each dinucleotide site based on dinucleotide sites before treatment and a corresponding plurality of treated dinucleotide sites.

Exemplarily, based on the principle of bisulfite treatment, the dinucleotide sites before and after treatment can be aligned to determine whether the original dinucleotide site is a methylated state.

In some embodiments, operation 3062 may be implemented in the following mode: aligning, for each first sample whole genome sequence, the dinucleotide site before treatment with a plurality of treated dinucleotide sites to obtain a plurality of alignment results corresponding to each dinucleotide site; determining that the methylation status of the dinucleotide site is an unmethylated state when the alignment result indicates that cytosine of the dinucleotide site is converted to uracil, or is not converted; determining that the methylation status of the dinucleotide site is a methylated state; and for each dinucleotide site when the alignment result indicates that cytosine of the dinucleotide site is converted to guanine, determining the actual methylation status of the dinucleotide site based on a second ratio of a methylated count of the dinucleotide site to a total occurrence count of the dinucleotide site, where the second ratio is a probability of methylation of the dinucleotide site. By aligning the original site before treatment with multiple treated versions of the same site (i.e., a plurality of sequencing rounds or technical replicates), the embodiment of this application significantly reduces the impact of single-experiment errors (e.g., PCR amplification bias, sequencing errors).

Exemplarily, since individual data lacks generalizability, the results of multiple bisulfite treatments for the same dinucleotide site are aggregated, and the methylated count and unmethylated count are calculated based on the alignment results. The total occurrence count of the dinucleotide site at the same position is the sum of the methylated count and the unmethylated count. A formula for calculating the second ratio is represented as follows

$${Methy}_{status} = \frac{{M}_{count}}{{M}_{count} + N {M}_{count}} ,$$

where M_{count} is the methylated count, and NM_{count} is the unmethylated count.

In the embodiment of this application, the actual methylation status is determined through experiments, providing more accurate annotation data for subsequent training to improve training accuracy. By determining the methylation status based on a plurality of data points and characterizing the methylation status with a probability, the singleness of data is avoided, and the reliability of the training data is improved.

Continuing to refer to FIG. 3A, operation 302: Extract a target dinucleotide site from a plurality of dinucleotide sites of each first sample whole genome sequence, and take a sequence within a preconfigured window range including the target dinucleotide site as a second sample whole genome sequence.

Exemplarily, the second sample whole genome sequence is a whole genome sequence obtained by cropping, based on the target dinucleotide site as a reference site, from the complete first sample whole genome sequence. A cropping range is a preconfigured window range, which may be determined according to actual scenarios.

In some implementations, referring to FIG. 3C, FIG. 3C is a third schematic flowchart of a method for training a prediction model provided by an embodiment of this application. Operation 302 may be implemented through operation 3021 in FIG. 3C, as described in detail below.

Exemplarily, the dinucleotide site is acquired by whole genome sequencing, which has been described above and will not be repeated here. The target dinucleotide site may be at least one of a plurality of dinucleotide sites, or all of them.

Operation 3021: Intercept, with the target dinucleotide site as a reference site, a sequence within the preconfigured window range from the first sample whole genome sequence as the second sample whole genome sequence.

Here, the reference site is located at any one of the following positions in the preconfigured window range: a boundary of the preconfigured window range, or an interior of the preconfigured window range.

Exemplarily, for example, if the target dinucleotide site is at a certain position in the first sample whole genome sequence, a sequence with a window width W is intercepted from the first sample whole genome sequence centering on that position as the second sample whole genome sequence.

In the embodiment of this application, by limiting the length of the training samples, the computational resources consumed during the training process can be saved, and the specificity of the prediction processing of the prediction model can be improved. By limiting the window width, the amount of data that the prediction model needs to process can be reduced, thereby accelerating the training process and improving efficiency. An appropriate window width can help the prediction model find a balance between memorizing patterns in the genome sequence and generalizing to new data.

Continuing to refer to FIG. 3A, operation 303: Annotate an actual methylation status for the target dinucleotide site in each second sample whole genome sequence to obtain an annotated second sample whole genome sequence.

Exemplarily, the mode of acquiring the actual methylation status has been described above in operations 3061 to 3062, which will not be repeated here. For each second sample whole genome sequence, the position of the target dinucleotide site in the second sample whole genome sequence is annotated, and the actual methylation status of the target dinucleotide site is annotated. The actual methylation status is characterized by a numerical value ranging from 0 to 1, where a value closer to 1 indicates that the target dinucleotide site is more likely to be in a methylated state.

Exemplarily, for the actual methylation status annotated for each target dinucleotide site, the actual methylation status and the second sample whole genome sequence are combined into a second training dataset.

In some embodiments, the second training dataset is a training set for the second prediction model. A validation set and a test set for the second prediction model may also be created based on the real data corresponding to the first training dataset. The validation set is a dataset configured for adjusting model hyperparameters and evaluating model performance. The test set is a dataset configured for evaluating the performance of a trained model. After model training is completed, the test set is configured to validate the generalization capability of the model on unseen data. The performance of the model on the test set can help evaluate the accuracy and performance of the model.

Operation 304: Perform training processing on the second prediction model based on each annotated second sample whole genome sequence to obtain a trained third prediction model.

Here, the third prediction model is configured to predict a methylation status of a dinucleotide site in a to-be-detected whole genome sequence.

Exemplarily, the training processing is configured to train the function of the second prediction model for the methylation status of a dinucleotide site in a to-be-detected whole genome sequence. The training processing may be performed iteratively, and the loss function used may be cross-entropy loss or binary cross-entropy loss.

In some embodiments, referring to FIG. 3E, FIG. 3E is a fifth schematic flowchart of a method for training a prediction model provided by an embodiment of this application. Operation 304 may be implemented through operations 3041 to 3044 in FIG. 3E, as described in detail below.

Operation 3041: Call the second prediction model to perform the following processing: performing feature extraction processing on each second sample whole genome sequence to obtain a second feature vector of each second sample whole genome sequence.

Exemplarily, the feature extraction processing may refer to operation 3011 described above.

The purpose of operation 3041 is to extract meaningful, low-dimensional numerical representations (i.e., second feature vectors) from the original genomic data using an existing second prediction model. The goal of this operation is to convert an original, high-dimensional, complex whole genome sequence (a long string includes A, T, C, G bases) into a fixed-length numerical vector (a second feature vector) containing rich information. This provides a more easily processable and informative input for the subsequent construction of the second prediction model.

The input is a plurality of second sample whole genome sequences, which are the original input data, and each sample represents a complete genome sequence (e.g., DNA from different individuals or cells).

Internally, when a whole genome sequence is input into the model, the model processes it layer by layer: The bottom layers may recognize local patterns (e.g., specific k-mer combinations), the middle layers may combine these local patterns to identify more complex structures or contextual relationships, and before the final layers (or a specifically designed output layer), the model internally forms a high-dimensional, abstract internal representation of the input sequence. This internal representation (typically the activation values of a certain layer near the end of the model or of a specific pooling layer) is extracted as the “second feature vector” for the sample, and the second feature vector is finally output.

For each input second sample whole genome sequence, after being processed by the second prediction model, a corresponding fixed-length numerical vector is output. This vector is no longer the original base sequence, but a compressed, abstract representation of the overall or key characteristics of the sequence learned by the model. It contains information that the model considers useful for understanding the sequence structure and potential biological significance (even if methylation is not directly involved at this point).

Through operation 3041, a corresponding fixed-dimensional feature vector (second feature vector) is generated for each input whole genome sequence. These vectors are more compact and information-dense than the original sequences, providing a high-quality input foundation for the subsequent construction of a second prediction model that focuses more on specific tasks (e.g., predicting methylation).

Operation 3042: Determine, based on the second feature vector of each second sample whole genome sequence, a first prediction probability that the target dinucleotide site included in each second sample whole genome sequence is in a methylated state.

In some embodiments, operation 3042 may be implemented by performing the following processing for each second sample whole genome sequence: performing multi-level feature conversion processing on the second feature vector to obtain a converted second feature vector, where the feature conversion processing includes feature extraction based on a transformer block and column pooling processing; calling an attention mechanism to perform aggregation processing based on the converted second feature vector to obtain a global feature; and performing activation processing based on the global feature of the second sample whole genome sequence to obtain a first prediction probability that the target dinucleotide site is in a methylated state.

In some embodiments, the second prediction model includes an embedding layer, a plurality of transformer blocks, and a prediction head, where the embedding layer is configured to perform feature extraction processing on a whole genome sequence, the plurality of transformer blocks are configured to perform multi-level feature conversion processing, and the prediction module is configured to perform aggregation processing and activation processing. The second prediction model, the first prediction model, and the third prediction model are names for different stages of the same model, and the first prediction model and the third prediction model have the same structure as the second prediction model.

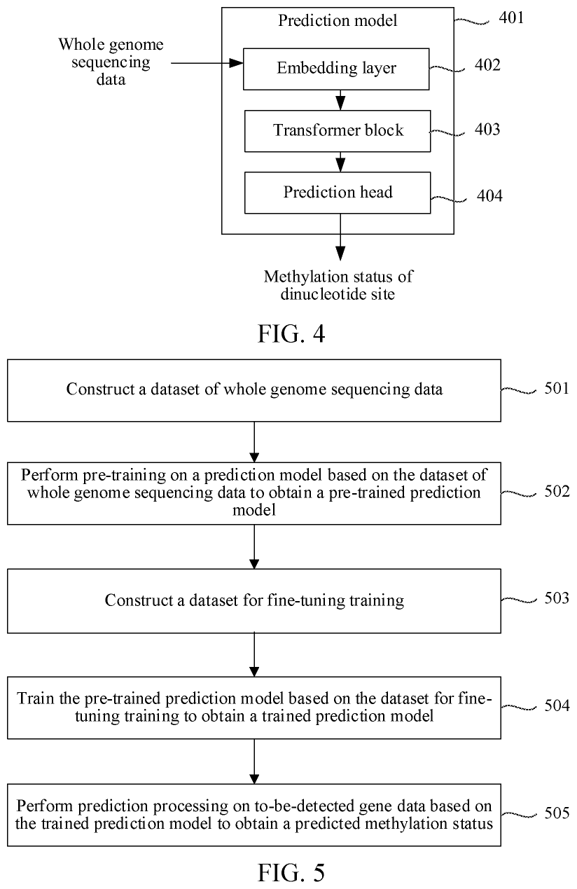

Exemplarily, referring to FIG. 4, FIG. 4 is a first schematic structural diagram of a prediction model provided by an embodiment of this application. The prediction model 401 includes an embedding layer 402, a transformer block 403, and a prediction head 404. The prediction head further integrates global information through a global attention mechanism. The global attention mechanism enhances the semantic understanding capability of the model by calculating correlations among all positions. The prediction head includes a plurality of linear layers and activation functions, such as SiLU and Linear, configured to generate a final result.

Through the cascaded design of local feature conversion (transformer+column pooling)→global attention aggregation→probability activation, the embodiment of this application achieves high-precision methylation status prediction and robust modeling across tissues and species.

Operation 3043: Determine a second loss function of the second prediction model based on a labeling probability corresponding to the actual methylation status of each target dinucleotide site and the first prediction probability.

Exemplarily, the second loss function is a mean of sub-losses corresponding to each target dinucleotide site, and the sub-loss may be cross-entropy loss or binary cross-entropy loss. In the embodiment of this application, binary cross-entropy loss is used as an example for description.

In some embodiments, operation 3043 may be implemented in the following mode: determining, for each target dinucleotide site, a binary cross-entropy loss between the labeling probability corresponding to the actual methylation status and the first prediction probability; and taking a mean of a plurality of the binary cross-entropy losses as the second loss function of the second prediction model.

Exemplarily, the binary cross-entropy loss may be represented by the following formula (1):

L=−[y log y′+(1−y)log(1−y′)]
where L is the binary cross-entropy loss, y is the labeling probability corresponding to the actual methylation status, and y′ is the first prediction probability.

Through the combination of probabilistic labeling and mean aggregation of binary cross-entropy loss, and by strictly aligning the gradient update direction with biological significance, the embodiment of this application achieves stronger noise robustness: mean aggregation shields local anomalies, ultimately improving the prediction accuracy of the methylation prediction model in complex scenarios.

Operation 3044: Perform iterative parameter update processing on the second prediction model based on the second loss function to obtain a third prediction model.

Exemplarily, the third prediction model is configured to determine a methylation status of a dinucleotide site in a whole genome sequence. The number of iterations and the learning rate used in the parameter update processing may be set according to actual application scenarios. The mode of parameter update processing is backpropagation processing.

In the embodiment of this application, by limiting the range of training data, data complexity is reduced, training efficiency is improved, and computational resources required for the training process are saved. Dividing the training into two stages, i.e., pre-training and fine-tuning training, can improve the generalization capability and accuracy of the trained model.

In some embodiments, after operation 304, the following processing is performed: acquiring a third training dataset, where the third training dataset includes: a plurality of third whole genome sequences and sample label probabilities of sample dinucleotide sites respectively corresponding to the third whole genome sequences; calling the third prediction model to perform prediction processing based on the plurality of third whole genome sequences to obtain a second prediction probability that a sample dinucleotide site in each third whole genome sequence is in a methylated state; determining a first difference between the sample label probability and the second prediction probability (corresponding to the sample dinucleotide site), and determining a first number of the first differences that are less than a preconfigured difference; and taking, in response to an accuracy of the third prediction model being less than a preconfigured accuracy, the third prediction model as the second prediction model, and proceeding to operation 304.

Exemplarily, the accuracy is a ratio of the first number to a total number of the sample dinucleotide sites. A first difference being less than a preconfigured difference indicates that the second prediction probability predicted by the third prediction model is close to the sample label probability. Thus, the first number of the first differences can be configured to characterize the accuracy of the third prediction model. The accuracy is detected through the validation set. If the accuracy is relatively low, the prediction model is retrained. The third training dataset and the first training dataset are extracted from the same dataset at a specific ratio, and the specific ratio may be set according to actual application scenarios. The first training dataset serves as a training set, and the third training dataset serves as a validation set during the training process.

The embodiment of this application innovatively combines probabilistic continuity evaluation with dynamic model degradation, achieving three major breakthroughs: refined evaluation: using probability difference instead of binary accuracy to reveal the true performance of the model within confidence intervals; intelligent iteration: automatically triggering model rollback based on quantitative indicators, avoiding resource waste.

In some embodiments, after operation 304, the following processing is performed: calling the third prediction model based on a to-be-detected whole genome sequence to perform the following processing: performing feature extraction processing on the to-be-detected whole genome sequence to obtain a fourth feature vector of the to-be-detected whole genome sequence; determining a third prediction probability that a to-be-detected dinucleotide site included in the second sample whole genome sequence is in a methylated state based on the fourth feature vector; and determining that the to-be-detected dinucleotide site is in a methylated state in response to the third prediction probability being greater than a preconfigured probability.

Exemplarily, the preconfigured probability may be set according to actual application scenarios. The value range of the preconfigured probability is a positive number less than 1. When the third prediction probability is greater than the preconfigured probability, it indicates that the to-be-detected dinucleotide site is in a methylated state.

The setting of the preconfigured probability (decision threshold) needs to balance prediction accuracy and application scenario requirements. Its essence is to draw a demarcation line between the continuous methylation probability (third prediction probability) output by the model and the binary classification decision (methylated/unmethylated). Optimization may be performed based on validation set performance by: first, generating a probability distribution, inputting an independent validation set (including dinucleotide sites with known methylation status) into the third prediction model, and acquiring the predicted probability for each site (third prediction probability); then, drawing evaluation curves, where an ROC curve is configured to calculate the true positive rate (TPR) and false positive rate (FPR) under different thresholds, and a PR curve (applicable to imbalanced sample scenarios) is configured to calculate precision and recall; selecting an optimal threshold to maximize a first metric (TPR-FPR, a point corresponding to a top-left corner of the ROC curve), or maximizing a F1 score: F1=2×(Precision×Recall)/(Precision+Recall) (a peak point of the PR curve); and if high specificity (e.g., clinical diagnosis) is required, selecting a high threshold (e.g., 0.95) to reduce false positives, and if high sensitivity (e.g., screening) is required, selecting a low threshold (e.g., 0.7) to reduce false negatives.

In the embodiment of this application, the trained model can be configured to detect the methylation status of a whole genome sequence. Compared with solutions in the related art that determine the methylation status of a whole genome sequence through experimental means, there is no need to perform complex experiments, and the efficiency and accuracy of methylation status detection are improved.

In some embodiments, the training data used in the method for training a prediction model provided by the embodiment of this application includes, but is not limited to, various types of biological data such as RNA sequencing data (RNA-seq) and chromatin immunoprecipitation sequencing data (ChIP-seq). By integrating a plurality of types of biological data, the prediction model can capture methylation information in the genome from different levels, thereby improving the accuracy and coverage of prediction.

In the embodiments of this application, through pre-training, the prediction model is enabled to learn relevant features in whole genome sequences. In the training phase, whole genome sequences within a specific range are configured to perform targeted training, thereby improving the accuracy of the prediction model in predicting methylation status, by training a prediction model for methylation status. By limiting the range of training data, data complexity is reduced, training efficiency is improved, and computational resources required for the training process are saved. By detecting a to-be-detected genome sequence using the trained prediction model, compared with methylation status detection in the related art, there is no need to perform complex experiments, noise and interference introduced during the experimental process are avoided, and the efficiency and accuracy of methylation status detection are improved.

Below, an exemplary application of the method for training a prediction model according to the embodiment of this application in a practical application scenario will be described.

Methylation is an epigenetic modification phenomenon that mainly involves adding a methyl group (—CH₃) to a DNA molecule, typically occurring at the 5^{th} carbon position of cytosine. Methylation is an important epigenetic regulation mechanism that has a significant influence on biological processes such as gene expression, gene silencing, genome stability, chromatin structure, and development. An abnormal methylation status is closely related to various diseases, such as cancer, neurological diseases. Existing whole-genome bisulfite sequencing (WGBS) can obtain methylation information across the entire genome. The whole-genome bisulfite sequencing method has high resolution and comprehensive coverage, but has high experimental costs and requires complex data processing.

Solutions in the related art can be classified into two categories: (1) prediction methods based on base pair arrangement patterns of sequences; and (2) prediction methods based on sequencing of methylation site sequences.

For solution (1), i.e., prediction methods based on base pair arrangement patterns of sequences, such methods predict methylation status by analyzing the base sequence patterns of DNA sequences. By analyzing plasma samples from healthy individuals and cancer patients, fragment patterns of cell-free DNA (cfDNA) are analyzed using whole genome sequencing (WGS) and whole-genome bisulfite sequencing (WGBS). Studies have found that methylated dinucleotide sites (CpG dinucleotide sites), i.e., CG sites, have a higher probability of cleavage at the CGN end and a lower probability of cleavage at the NCG end. Therefore, the CGN/NCG ratio is higher at hypermethylated dinucleotide sites and lower at hypomethylated dinucleotide sites. By analyzing these ratios, the methylation level of a target sequence can be predicted. A CpG-end break refers to a break occurring at a dinucleotide site, i.e., a break occurring at the end of cytosine or guanine. Such a break may affect the methylation status of the dinucleotide site, especially methylation changes occurring near the break point. An NCG-end break (non-CpG-end break) refers to a break occurring on a non-dinucleotide sequence adjacent to a dinucleotide site. Such a break may not directly affect the methylation status of the dinucleotide site, but it may affect the overall structure and stability of the genomic region in which the dinucleotide site is located.

For solution (2), i.e., prediction methods based on sequencing of methylation site sequences, such methods predict methylation status by directly sequencing the sequences of methylation sites in a DNA sample. By collecting plasma samples from healthy individuals and patients with various types of cancer, performing whole-genome bisulfite sequencing (WGBS) and whole genome sequencing (WGS) on cell-free DNA, and creating the FinaleMe model, the methylation status in cell-free DNA fragment patterns is predicted using an inhomogeneous hidden Markov model (NHMM) and a Gaussian mixture model (GMM). The FinaleMe model can accurately predict methylation status, and its predictive capability has been validated in various cancer types, demonstrating its potential in non-invasive cancer diagnosis.

However, the related art has the following problems: (1) High experimental complexity and high costs: solutions in the related art, such as prediction methods based on base pair arrangement patterns of sequences and prediction methods based on sequencing of methylation site sequences, typically require complex experimental procedures and high sequencing costs. In particular, whole-genome bisulfite sequencing (WGBS) requires bisulfite treatment of DNA and high-throughput sequencing, which increases experimental complexity and costs. (2) Complex data processing: sequencing data processing in the related art requires complex bioinformatics analysis, including operations such as data preprocessing, feature extraction, and model training. These operations require not only high-performance computing resources but also specialized bioinformatics knowledge and skills, limiting the popularization and adoption of these technologies in practical applications. (3) Limited prediction accuracy: the related art relies on sequence features or specific methylation sites and may not be able to fully capture changes in methylation status across the entire genome, resulting in limited prediction accuracy and coverage. In addition, due to the presence of noise and technical deviations, existing methods may have a relatively high error rate when processing complex biological samples.

The embodiment of this application provides a method for training a prediction model. By performing pre-training using whole genome sequencing (WGS) data and performing fine-tuning using paired WGS data and methylation status of CG sites (dinucleotide sites) to predict the methylation status of gene sites, it can solve the following problems of the related art by: (1) Reducing experimental complexity and costs: methylation-specific experiments are not required, and the methylation status can be predicted using only WGS data, significantly reducing experimental complexity and costs. (2) Simplifying the data processing workflow: by utilizing a large-scale genomics model, the data processing workflow is simplified, prediction accuracy and efficiency are improved, and reliance on high-performance computing resources and specialized skills is reduced. (3) Improving prediction accuracy and coverage: changes in methylation status across the entire genome can be fully captured. By integrating WGS data and advanced machine learning techniques, the accuracy and coverage of methylation status prediction are significantly improved, effectively addressing noise and technical deviations in complex biological samples.

The method for training a prediction model provided by the embodiment of this application will be explained and described below with reference to the accompanying drawings. Referring to FIG. 5, FIG. 5 is a sixth schematic flowchart of a method for training a prediction model provided by an embodiment of this application. The execution subject of the operations in FIG. 5 is the server 200 in FIG. 1.

Operation 501: Construct a dataset of whole genome sequencing data.

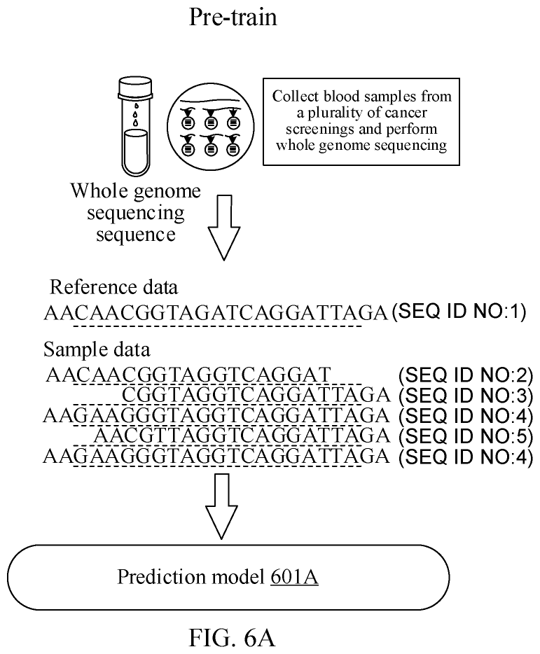

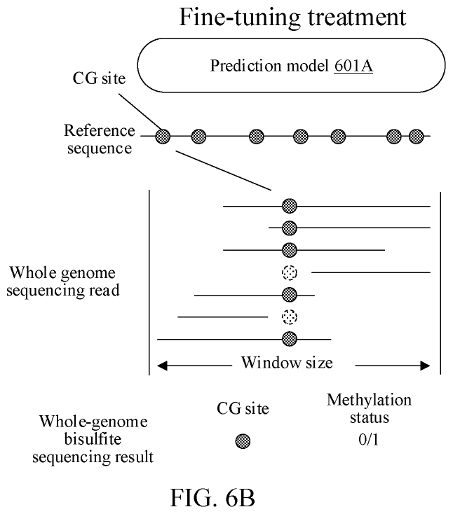

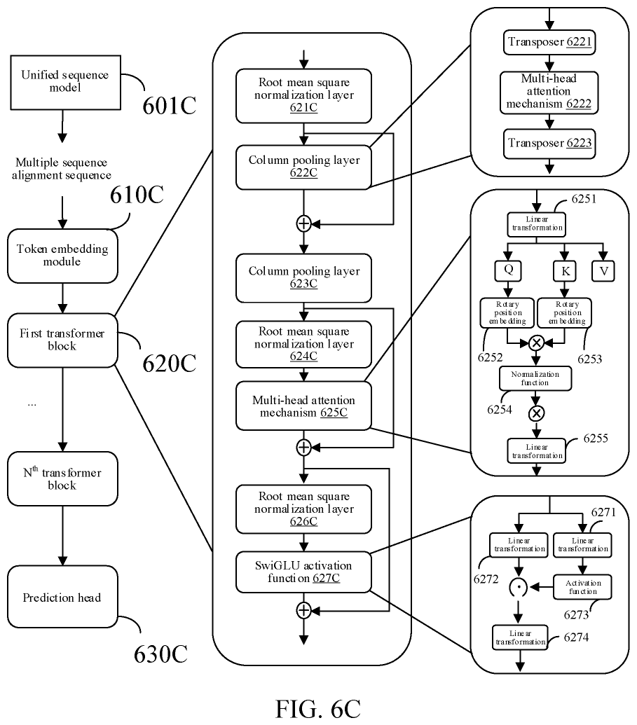

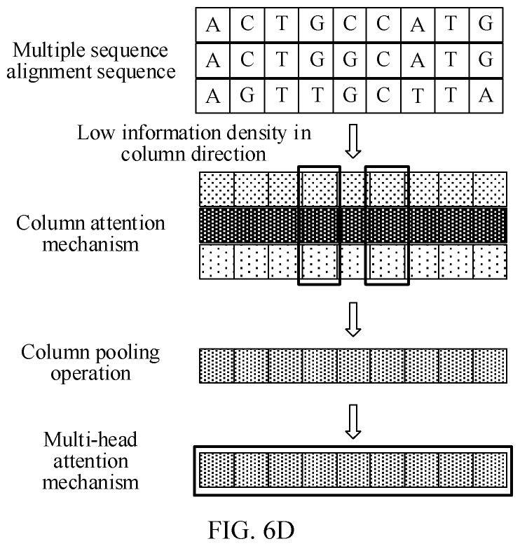

Exemplarily, referring to FIG. 6A, FIG. 6A is a first schematic diagram of a training principle provided by an embodiment of this application. Blood samples are collected from a plurality of cancer screenings and whole genome sequencing (WGS) is performed to obtain whole genome sequence data of the samples. A reference sequence (simulated data) and the WGS sequences of the samples (real data) are combined to construct a training dataset.

The whole genome sequencing data shown in FIG. 6A includes reference data and sample data. Description will be made by taking the reference data as an example. The content of the reference data is “ . . . . AACAACGGTAGATCAGGATTAGA . . . ” (SEQ ID NO:1). DNA includes two long strands that are intertwined in the form of a double helix. DNA has four types of nitrogenous bases: adenine (A), thymine (T), cytosine (C), and guanine (G). At corresponding positions on the two long strands, adenine always pairs with thymine, and cytosine always pairs with guanine. Therefore, by determining the base sequence of one long strand of DNA, the base sequence of the other long strand of DNA can be determined. Both the reference data and the sample data are represented in the form of a single long strand, and the base sequence of the other long strand can be determined based on the base sequence of the shown single long strand.

In some embodiments, bioinformatics methods are configured to extract biological features from whole genome sequencing data, such as CG site (dinucleotide site) content, repetitive sequences, genomic domains. These features are used as input to train a machine learning model (e.g., a support vector machine or a random forest model) for methylation status prediction. Existing biological knowledge may be utilized to improve the interpretability of the prediction.

In the embodiment of this application, whole genome sequencing (WGS) data is configured to predict the methylation status of gene sites, improving prediction accuracy. At the same time, by performing detection on WGS data using the model, methylation-specific experiments are not required, and the methylation status can be predicted using only WGS data, thereby reducing experimental complexity and costs.

Operation 502: Perform pre-training on a prediction model based on the dataset of whole genome sequencing data to obtain a pre-trained prediction model.

Exemplarily, the method for training a prediction model provided by the embodiment of this application includes two stages, i.e., pre-training and fine-tuning. In the pre-training stage, preliminary training of a deep learning model is performed on a large amount of whole genome sequencing (WGS) data (represented in the form of sequences) to extract potential features and patterns from genome sequences. In the fine-tuning stage, specific methylation site data is used to further train the model to optimize and improve the accuracy and reliability of methylation status prediction. The aim is to reduce experimental complexity and costs, while providing an efficient and accurate solution for DNA methylation status prediction.

Continuing to refer to FIG. 6A, the prediction model 601A is a deep learning architecture. The prediction model 601A includes layers such as feed forward, column attention, row attention, and layer normalization. The pre-training mode may be self-supervised learning training, so that the model can generate feature vectors describing input genome sequences. During the model training process, the model implicitly learns coverage information of WGS sequences, CGN/NCG ratio information of CG sites, fragment length information of each sequencing read, and sequence information at both ends of sequencing fragments.

In some embodiments, the prediction model 601A may be a combination of multiple different types of models, such as deep learning models, conventional machine learning models, statistical models, working together to perform methylation status prediction. Ensemble learning can fully leverage the advantages of different models, reduce prediction errors, and improve the stability and accuracy of prediction.

Operation 503: Construct a dataset for fine-tuning training.

Exemplarily, a human reference genome is traversed to acquire chromosome and position information of all CG sites for subsequent dataset construction. For example, blood samples are collected from a plurality of cancer screenings, and whole genome sequencing (WGS) and whole-genome bisulfite sequencing (WGBS) are performed to identify CG sites in the reference genome. Whole genome sequencing is a technology capable of determining the entire DNA sequence of an individual's whole genome. Sequencing reads are aligned with a reference genome to determine the nucleotide sequence at each site. Whole-genome bisulfite sequencing is a method for determining DNA methylation status. Its principle is to convert unmethylated cytosine (C) to uracil (U) through bisulfite treatment, followed by sequencing. A methylated dinucleotide site in the original DNA will appear as GpG, while an unmethylated dinucleotide site will appear as CpG or UpG. Thereafter, the methylation status of a dinucleotide site can be determined through sequencing and alignment analysis. By aligning the sequenced reads with the reference genome, the methylation status of a dinucleotide site is determined based on U (indicating that C at the original dinucleotide site has been converted to U) and G (indicating the original GpG site).

Referring to FIG. 6B, FIG. 6B is a second schematic diagram of a training principle provided by an embodiment of this application. Since a CG site in each sequence may exhibit different methylation statuses and individual data lacks generalizability, the methylation status of each CG site can be calculated based on a plurality of detection results, and the reference genome sequence of the CG site within a defined window size W is combined with the whole genome sequencing data to obtain a training sample, thereby constructing a training dataset. The training label of the training sample is the methylation status of the CG site in the whole-genome bisulfite sequencing results.

Constructing a training dataset using whole genome sequencing results of CG sites may be implemented in the following mode: selecting a hyperparameter window width W (the window width defines the range of the genomic region to be analyzed, and a specific sequence length may be set as the window width according to actual application scenarios) to acquire whole genome sequencing information around the CG site, while recording corresponding information such as chromosome, position, methylation status. For each CG site, the methylation status Methyₛₜₐₜᵤₛ of the site is calculated using the methylated count and unmethylated count of the CG site in the whole-genome bisulfite sequencing report. The specific calculation method is represented as

$${Methy}_{status} = \frac{{M}_{count}}{{M}_{count} + N {M}_{count}} ,$$

where M_{count} is the methylated count, and NM_{count} is the unmethylated count.

In some embodiments, the datasets of all samples are merged and divided according to division criteria to construct a training set, a validation set, and a test set. The training set corresponds to the first training dataset and the second training dataset described above, and the validation set corresponds to the third training dataset described above.

The training set is a dataset configured for training a machine learning model. During training, the model uses samples in the training set to learn features and patterns in order to make predictions or classifications.

The validation set is a dataset configured for adjusting model hyperparameters and evaluating model performance. It is a sample set separately reserved during the model training process, and can be configured to adjust model hyperparameters and to perform preliminary evaluation of model capability. It is typically used during iterative model training to validate the generalization capability (accuracy, recall, etc.) of the current model, so as to determine whether to continue training. In neural networks, the validation dataset is configured to find the optimal network depth (number of hidden layers), or to determine the stopping point of a backpropagation algorithm, or to select the number of neurons in hidden layers in the neural networks. In conventional machine learning, cross-validation, which is commonly used, involves subdividing the training dataset itself into different validation datasets to train the model.

The test set is a dataset configured for evaluating the performance of a trained model. After model training is completed, the test set is configured to validate the generalization capability of the model on unseen data. The performance of the model on the test set can help evaluate the accuracy and performance of the model.

Operation 504: Train the pre-trained prediction model based on the dataset for fine-tuning training to obtain a trained prediction model.

Exemplarily, paired whole genome sequencing data and methylation status of CG sites are configured to fine-tune the model, with a focus on learning and detecting the influence of features such as the position of CG sites in whole genome sequencing sequences, the sequencing depth of whole genome sequencing sequences, and the fragmentation pattern of whole genome sequencing sequences on methylation status. The loss function used during the training process may be cross-entropy loss or binary cross-entropy loss. During the model training process, the model implicitly learns coverage information of whole genome sequencing sequences, CGN/NCG ratio information of CG sites, fragment length information of each sequencing read, and sequence information at both ends of sequencing fragments, and uses such information to predict the methylation status of the site.

Exemplarily, the sequencing depth of a whole genome sequencing sequence refers to the average number of times (frequency) each base is detected during the whole genome sequencing process. The calculation of depth information is typically based on reads generated by sequencing and alignment results at corresponding positions on a reference genome. The total coverage of each site or region may be calculated by summing the number of all reads covering the site or region, and then dividing by the number of base pairs in the site or region. For example, if a gene has 100 base pairs and a total of 1000 reads cover this region, then the coverage depth of the gene is 10 (1000 reads/100 bases). The fragmentation pattern of a whole genome sequencing sequence is configured to describe structural variations such as large fragment deletions, insertions, inversions, or duplications in the genome.

To facilitate understanding, the structure of the prediction model will be explained and described below with reference to FIG. 6C. FIG. 6C is a second schematic structural diagram of a prediction model provided by an embodiment of this application. The input of the prediction model is a multiple sequence alignment (MSA) sequence, and the output is predicted methylation status information. The complete model structure is divided into five parts. The multiple sequence alignment sequence may be provided by a unified sequence model (USM) 601C. The prediction model includes a token embedding module 610C, a plurality of transformer blocks (e.g., a first transformer block 620C and an N^{th} transformer block, where N is a positive integer), and a prediction head 630C. Input raw sequence data (MSA/sequence) undergoes token embedding processing by the token embedding module 610C, which maps each token in each sequence to a high-dimensional vector space. The converted input data then passes through the N transformer blocks.

The structure of a transformer block will be explained and described by taking the first transformer block 620C as an example. According to the data flow direction, the first transformer block 620C includes a root mean square normalization layer 621C, a column pooling layer 622C, a column pooling layer 623C, a root mean square normalization layer 624C, a multi-head attention mechanism 625C, a root mean square normalization layer 626C, and a SwiGLU activation function 627C.

The root mean square normalization layer 621C is configured to perform root mean square normalization (RMS Norm) processing on input data, calculate the mean of the squares of the gradients of each feature, and then use this mean as a scaling factor for normalization. When using residual connections, root mean square normalization can better handle differences between different dimensions because it does not depend on a specific network architecture. The process of root mean square normalization includes: 1) calculating the square of the gradient of each feature; 2) calculating the mean of the squares of the gradients of all features; and 3) performing normalization processing. Normalization is a common data preprocessing technique configured to convert data into a uniform scale range to eliminate dimensional differences between different variables. Through normalization, data can be mapped to a specific range, such as [0, 1] or [−1, 1].

In the column pooling layer, taking the column pooling layer 622C as an example for description, the column pooling layer 622C includes a transposer 6221, a multi-head attention mechanism 6222, and a transposer 6223. The column pooling layer is configured to reduce the dimensionality of data while retaining important information by aggregating information of each column.

The multi-head attention mechanism is configured to calculate correlations between different parts of input data. The multi-head attention mechanism includes linear transformation, attention mechanism, rotary position embedding (RoPE), normalization function softmax, and linear transformation. Continuing to refer to FIG. 6C, the multi-head attention mechanism 625C is configured to perform processes such as linear transformation 6251, rotary position embedding 6252, rotary position embedding 6253, normalization function 6254, and linear transformation 6255. Rotary position embedding (RoPE) is a method for encoding position information. Absolute position encoding assigns a unique vector to each position. Although simple, it does not scale well and cannot effectively capture relative positions. Relative position encoding focuses on distances between tokens, enhancing the model's understanding of token relationships, but complicates the model architecture. Rotary position embedding combines the advantages of both. It allows position information to be encoded in a mode that enables the model to understand both the absolute positions of tokens and their relative distances. This is achieved through a rotation mechanism, where each position in the sequence is represented by a rotation in the embedding space. The advantage of rotary position embedding lies in its simplicity and efficiency, which enables the model to better capture the nuances of language grammar and semantics.

The SwiGLU activation function 627C in the first transformer block 620C is configured to perform the following processing: linear transformation 6271, linear transformation 6272, activation function 6273, and linear transformation 6274. The Sigmoid gated linear unit (SwiGLU) activation function can replace the conventional rectified linear unit (ReLU) activation function to increase the expressive power of the model.

In some embodiments, the transformer block further includes a residual connection module configured to add input data to transformed data, preventing gradient vanishing (gradients becoming very small) or explosion (gradients becoming very large). The basic idea of a residual connection is to introduce an additional connection between each layer of the network, so that the output of the network can directly add the input without undergoing non-linear transformation. In this way, even if the non-linear transformation causes distortion of the gradient during propagation, the input data itself can be directly passed to the next layer, thereby preserving gradient information to some extent.

In some embodiments, the transformer block further includes a layer normalization layer configured to perform normalization processing on transformed data. Layer normalization calculates the mean and variance of each feature in each sample, and then normalizes the feature values of each sample to a distribution with a mean of 0 and a standard deviation of 1. Layer normalization helps stabilize training, particularly in deep networks using residual connections, as it can reduce internal covariate shift. Layer normalization is typically applied to all features of each sample, and thus it takes into account both re-scaling and re-centering invariance.

The prediction head 630C is configured to further integrate global information through a global attention mechanism. The global attention mechanism enhances the semantic understanding capability of the model by calculating correlations among all positions. The prediction head 630C is further configured to perform a final classification or regression task. The prediction head includes a plurality of linear layers and activation functions, such as SiLU and Linear, configured to generate a final result. The entire training process optimizes model parameters through a backpropagation algorithm, so that the performance of the model on the training data gradually improves.

Referring to FIG. 6D, FIG. 6D is a schematic diagram of a data flow of a prediction model provided by an embodiment of this application. A multiple sequence alignment sequence has low information density in the column direction. The column attention mechanism, column pooling operation, and multi-head attention mechanism are sequentially performed on it to acquire the methylation status.

In some embodiments, when training the prediction model, a plurality of tasks related to the methylation status, such as predicting sequence length, sequence breakpoint features, sequencing depth, may be considered simultaneously. By sharing part of the structure and parameters of the model, multi-task learning can improve the generalization capability of the model on various tasks, thereby improving the accuracy of methylation status prediction.

Continuing to refer to FIG. 5, operation 505: Perform prediction processing on to-be-detected gene data based on the trained prediction model to obtain a predicted methylation status.

Exemplarily, the trained model may take WGS sequencing data containing a CG site as input, and output a predicted methylation status value for the site. The predicted methylation status value is typically a probability value representing the probability that a given site is methylated (methylated state) or unmethylated (unmethylated state). For example, a prediction model may output a probability value between 0 and 1, where 0 represents completely unmethylated and 1 represents completely methylated.

Exemplarily, in practical applications, the data input into the trained model may also be RNA sequencing data (RNA-seq) or chromatin immunoprecipitation sequencing data (ChIP-seq). Chromatin immunoprecipitation sequencing data (ChIP-seq) is biological data obtained through chromatin immunoprecipitation (ChIP) technology combined with high-throughput sequencing. This technology is mainly configured to study the interactions between proteins (transcription factors, histone modification enzymes, etc.) and DNA. By integrating multi-modal data, the prediction model can capture methylation information in the genome from different levels, thereby improving prediction accuracy and coverage.

The embodiment of this application may be applied in the following application scenarios:
1. Prediction of methylation status of specific gene sites, which is an important research content in epigenetics and molecular biology. Accurately predicting methylation status helps a person skilled in the art gain an in-depth understanding of gene expression regulation mechanisms, explore the epigenetic etiology of diseases, and develop corresponding diagnostic and therapeutic strategies. Predicting the methylation status of gene sites related to diseases can help scientists understand the pathogenesis of diseases, identify biomarkers for diseases, and develop new treatment methods. Furthermore, the embodiment of this application may be extended to the prediction of other epigenetic marks, such as histone modification status. Histone methylation is one of the key mechanisms regulating gene expression. Predicting the methylation status of specific gene sites can help scientists understand how genes are activated or silenced, and how these changes affect cellular function.
2. Drug development: during the drug development process, predicting the methylation status of a drug target can help optimize drug design and improve drug efficacy.
3. Tumor research: predicting methylation status is of great significance in the research and clinical application of various diseases such as cancer and neurological diseases. It can be used not only for early screening and diagnosis of diseases, achieving the goals of early intervention and improved cure rates, but also for helping to identify epigenetic marks of specific diseases, providing a scientific basis for precision medicine and personalized treatment plans. In tumor research, predicting the methylation status of tumor-related genes can help identify potential driver genes and prognostic markers of tumors, providing guidance for tumor diagnosis and treatment.

The embodiment of this application, by using whole genome sequencing (WGS) data for pre-training and using paired WGS data and methylation status of CG sites for fine-tuning to predict the methylation status of gene sites, has significant beneficial effects. First, methylation-specific experiments are not required, and the methylation status can be predicted using only WGS data, significantly reducing experimental complexity and costs. By utilizing a large-scale genomics model, the data processing workflow is simplified, and prediction accuracy and efficiency are improved. Furthermore, it can fully capture changes in methylation status across the entire genome, improving prediction accuracy and coverage, and is suitable for complex biological samples. It is of great significance in the research and clinical application of various diseases such as cancer and neurological diseases, promoting the development of early screening and precision medicine for diseases. At the same time, it has broad scalability, is applicable not only to the prediction of methylation status but also to the prediction of other epigenetic marks, and has broad application prospects.

The following continues to describe an exemplary structure of an apparatus 455 for training a prediction model provided by the embodiment of this application implemented as software modules. In some embodiments, as shown in FIG. 2, the software modules in the apparatus 455 for training a prediction model stored in a memory 450 may include: a pre-training module 4551, configured to pre-train a first prediction model based on a plurality of first sample whole genome sequences to obtain a second prediction model; a sample acquisition module 4552, configured to extract a target dinucleotide site from a plurality of dinucleotide sites of each first sample whole genome sequence, and take a sequence within a preconfigured window range including the target dinucleotide site as a second sample whole genome sequence, the sample acquisition module 4552 being configured to annotate an actual methylation status for the target dinucleotide site in each second sample whole genome sequence to obtain an annotated second sample whole genome sequence, and the sample acquisition module 4552 being configured to combine each annotated second sample whole genome sequence into a second training dataset; and a fine-tuning training module 4553, configured to perform training processing on the second prediction model based on each annotated second sample whole genome sequence to obtain a trained third prediction model, the third prediction model being configured to predict a methylation status of a dinucleotide site in a to-be-detected whole genome sequence.

In some embodiments, the pre-training module 4551 is configured to call the first prediction model to perform feature extraction processing on a plurality of first sample whole genome sequences to obtain a first feature vector of each first sample whole genome sequence; determine a first loss function of the first prediction model based on a plurality of the first feature vectors; and perform iterative parameter update processing on the first prediction model based on the first loss function to obtain the second prediction model, where the second prediction model is configured to extract a feature vector of the to-be-detected whole genome sequence.

In some embodiments, the pre-training module 4551 is configured to perform, for each first sample whole genome sequence, the following processing: determining a first similarity between the first feature vector of the first sample whole genome sequence and a first feature vector of a third sample whole genome sequence, and determining a second similarity between the first feature vector of the first sample whole genome sequence and a first feature vector of a fourth sample whole genome sequence, where the third sample whole genome sequence is another first sample whole genome sequence having the same base sequence as the first sample whole genome sequence, and the fourth sample whole genome sequence is another first sample whole genome sequence having a different base sequence from the first sample whole genome sequence; and taking a first ratio of the first similarity to a first sum as a first sub-loss function corresponding to the first sample whole genome sequence, where the first sum is a sum of the first similarity and the second similarity; and taking a mean of a plurality of the first sub-loss functions as the first loss function of the first prediction model.

In some embodiments, the first sample whole genome sequence includes a plurality of dinucleotide sites; the sample acquisition module 4552 is configured to perform, for each first sample whole genome sequence, the following processing: extracting a target dinucleotide site from the plurality of dinucleotide sites of the first sample whole genome sequence; intercepting, with the target dinucleotide site as a reference site, a sequence within a preconfigured window range from the first sample whole genome sequence as the second sample whole genome sequence, where the reference site is located at any one of the following positions in the preconfigured window range: a boundary of the preconfigured window range, or an interior of the preconfigured window range.

In some embodiments, the fine-tuning training module 4553 is configured to call the second prediction model based on the second training dataset to perform the following processing: performing feature extraction processing on each second sample whole genome sequence in the second training dataset to obtain a second feature vector of each second sample whole genome sequence; determining, based on the second feature vector of each second sample whole genome sequence, a first prediction probability that the target dinucleotide site included in each second sample whole genome sequence is in a methylated state; determining a second loss function of the second prediction model based on a labeling probability corresponding to the actual methylation status of each target dinucleotide site and the first prediction probability; and performing iterative parameter update processing on the second prediction model based on the second loss function to obtain the third prediction model, where the third prediction model is configured to determine a methylation status of a dinucleotide site in a whole genome sequence.

In some embodiments, the fine-tuning training module 4553 is configured to perform, for each second sample whole genome sequence, the following processing: performing multi-level feature conversion processing on the second feature vector to obtain a converted second feature vector; calling an attention mechanism to perform aggregation processing based on the converted second feature vector to obtain a global feature; and performing activation processing based on the global feature of the second sample whole genome sequence to obtain the first prediction probability that the target dinucleotide site is in a methylated state.

In some embodiments, the fine-tuning training module 4553 is configured to determine, for each target dinucleotide site, a binary cross-entropy loss between the labeling probability corresponding to the actual methylation status and the first prediction probability; and take a mean of a plurality of the binary cross-entropy losses as the second loss function of the second prediction model.

In some embodiments, the sample acquisition module 4552 is configured to, before pre-training a first prediction model based on a plurality of first sample whole genome sequences to obtain a second prediction model, perform whole genome sequencing processing on a biological sample to obtain a plurality of real whole genome sequences; and take a preconfigured reference whole genome sequence and the plurality of real whole genome sequences as the first sample whole genome sequences.

In some embodiments, the first sample whole genome sequence includes a plurality of dinucleotide sites; the sample acquisition module 4552 is configured to, before extracting a target dinucleotide site from the plurality of dinucleotide sites of each first sample whole genome sequence and taking a sequence within a preconfigured window range including the target dinucleotide site as a second sample whole genome sequence, perform bisulfite treatment on each first sample whole genome sequence to obtain a plurality of treated first sample whole genome sequences, where each treated first sample whole genome sequence includes a plurality of treated dinucleotide sites; and determine an actual methylation status of each dinucleotide site based on dinucleotide sites before treatment and a corresponding plurality of treated dinucleotide sites.

In some embodiments, the sample acquisition module 4552 is configured to align, for each first sample whole genome sequence, the dinucleotide site before treatment with a plurality of treated dinucleotide sites to obtain a plurality of alignment results corresponding to each dinucleotide site; determine that the methylation status of the dinucleotide site is an unmethylated state when the alignment result indicates that cytosine of the dinucleotide site is converted to uracil, or is not converted; determine that the methylation status of the dinucleotide site is a methylated state when the alignment result indicates that cytosine of the dinucleotide site is converted to guanine; and determine, for each dinucleotide site, the actual methylation status of the dinucleotide site based on a second ratio of a methylated count of the dinucleotide site to a total occurrence count of the dinucleotide site, where the second ratio is a probability of methylation of the dinucleotide site.

In some embodiments, the fine-tuning training module 4553 is configured to, after performing training processing on the second prediction model based on each annotated second sample whole genome sequence to obtain a trained third prediction model, acquire a third training dataset, where the third training dataset includes a plurality of third whole genome sequences and sample label probabilities of sample dinucleotide sites respectively corresponding to the third whole genome sequences; call the third prediction model to perform prediction processing based on the plurality of third whole genome sequences to obtain a second prediction probability that a sample dinucleotide site in each third whole genome sequence is in a methylated state; determine a first difference between the sample label probability and the second prediction probability, and determine a first number of the first differences that are less than a preconfigured difference; and take, in response to an accuracy of the third prediction model being less than a preconfigured accuracy, the third prediction model as the second prediction model, and proceed to the operation of performing training processing on the second prediction model based on each annotated second sample whole genome sequence to obtain a trained third prediction model, where the accuracy is a ratio of the first number to a total number of the sample dinucleotide sites.

In some embodiments, the fine-tuning training module 4553 is configured to, after performing training processing on the second prediction model based on each annotated second sample whole genome sequence to obtain a trained third prediction model, call the third prediction model based on the to-be-detected whole genome sequence to perform the following processing: performing feature extraction processing on the to-be-detected whole genome sequence to obtain a fourth feature vector of the to-be-detected whole genome sequence; determining a third prediction probability that a to-be-detected dinucleotide site included in the second sample whole genome sequence is in a methylated state based on the fourth feature vector; and determining that the to-be-detected dinucleotide site is in a methylated state in response to the third prediction probability being greater than a preconfigured probability.

An embodiment of this application provides a computer program product, the computer program product including a computer-executable instruction, and the computer-executable instruction being stored in a computer-readable storage medium. A processor of an electronic device reads the computer-executable instruction from the computer-readable storage medium, and the processor executes the computer-executable instruction, causing the electronic device to execute the method for training a prediction model according to the embodiment of this application.

An embodiment of this application provides a computer-readable storage medium having a computer-executable instruction stored therein, the computer-executable instruction, when executed by a processor, causing the processor to execute the method for training a prediction model provided by the embodiment of this application, for example, the method for training a prediction model as shown in FIG. 3A.

In some embodiments, the computer-readable storage medium may be a memory such as an FRAM, a ROM, a PROM, an EPROM, an EEPROM, a flash memory, a magnetic surface memory, an optical disc, or a CD-ROM, or may be various devices including one or any combination of the memories.

In some embodiments, the computer-executable instruction may be written in a form of a program, software, a software module, a script, or code according to a programming language (including a compiler or interpreter language or a declarative or procedural language) in any form, and may be deployed in any form, including an independent program or a module, a component, a subroutine, or another unit suitable for use in a computing environment.

As an example, the computer-executable instruction may, but do not necessarily, correspond to a file in a file system, and may be stored in a part of a file that saves another program or other data. For example, the computer-executable instruction may be stored in one or more scripts in a hypertext markup language (HTML) file, stored in a file that is specially configured for a program in discussion, or stored in a plurality of collaborative files (for example, stored in files of one or more modules, subprograms, or code parts).

As an example, the computer-executable instruction may be deployed to be executed on an electronic device, or deployed to be executed on a plurality of electronic devices at the same location, or deployed to be executed on a plurality of electronic devices that are distributed in a plurality of locations and interconnected by using a communication network.

To sum up, in the embodiments of this application, through pre-training, the prediction model is enabled to learn relevant features in whole genome sequences. In the training phase, whole genome sequences within a specific range are configured to perform targeted training, thereby improving the accuracy of the prediction model in predicting methylation status, by training a prediction model for methylation status. By limiting the range of training data, data complexity is reduced, training efficiency is improved, and computational resources required for the training process are saved. By detecting a to-be-detected genome sequence using the trained prediction model, compared with methylation status detection in the related art, there is no need to perform complex experiments, noise and interference introduced during the experimental process are avoided, and the efficiency and accuracy of methylation status detection are improved.

What are described above are merely embodiments of this application and are not intended to limit the scope of protection of this application. Any modifications, equivalent substitutions, improvements, and the like made within the spirit and scope of this application shall be included within the scope of protection of this application.

What is claimed is:

1. A method for training a prediction model, the method being applied to an electronic device, the method comprising:
pre-training a first prediction model based on a plurality of first sample whole genome sequences to obtain a second prediction model;
extracting a target dinucleotide site from a plurality of dinucleotide sites of each first sample whole genome sequence, and taking a sequence within a preconfigured window range comprising the target dinucleotide site as a second sample whole genome sequence;
annotating an actual methylation status for the target dinucleotide site in each second sample whole genome sequence to obtain an annotated second sample whole genome sequence; and
performing training processing on the second prediction model based on each annotated second sample whole genome sequence to obtain a trained third prediction model, the third prediction model being configured to predict a methylation status of a dinucleotide site in a to-be-detected whole genome sequence.

2. The method according to claim 1, wherein the pre-training the first prediction model based on the plurality of first sample whole genome sequences to obtain the second prediction model comprises:
calling the first prediction model to perform feature extraction processing on the plurality of first sample whole genome sequences to obtain a first feature vector of each first sample whole genome sequence;
determining a first loss function of the first prediction model based on a plurality of first feature vectors; and
performing iterative parameter update processing on the first prediction model based on the first loss function to obtain the second prediction model, the second prediction model being configured to extract a feature vector of the to-be-detected whole genome sequence.

3. The method according to claim 2, wherein the determining the first loss function of the first prediction model based on the plurality of first feature vectors comprises:
performing, for each first sample whole genome sequence:
determining a first similarity between the first feature vector of the first sample whole genome sequence and a first feature vector of a third sample whole genome sequence, and determining a second similarity between the first feature vector of the first sample whole genome sequence and a first feature vector of a fourth sample whole genome sequence, the third sample whole genome sequence being another first sample whole genome sequence having a same base sequence as the first sample whole genome sequence, and the fourth sample whole genome sequence being another first sample whole genome sequence having a different base sequence from the first sample whole genome sequence;
taking a first ratio of the first similarity to a first sum as a first sub-loss function corresponding to the first sample whole genome sequence, the first sum being a sum of the first similarity and the second similarity; and
taking a mean of a plurality of first sub-loss functions as the first loss function of the first prediction model.

4. The method according to claim 1, wherein the taking the sequence within the preconfigured window range comprising the target dinucleotide site as the second sample whole genome sequence comprises:
intercepting, with the target dinucleotide site as a reference site, the sequence within the preconfigured window range from the first sample whole genome sequence as the second sample whole genome sequence, the reference site being located at any one of the following positions in the preconfigured window range: a boundary of the preconfigured window range, or an interior of the preconfigured window range.

5. The method according to claim 1, wherein the performing training processing on the second prediction model based on each annotated second sample whole genome sequence to obtain the trained third prediction model comprises:
calling the second prediction model to perform:
performing feature extraction processing on each second sample whole genome sequence to obtain a second feature vector of each second sample whole genome sequence;
determining, based on the second feature vector of each second sample whole genome sequence, a first prediction probability that the target dinucleotide site comprised in each second sample whole genome sequence is in a methylated state;
determining a second loss function of the second prediction model based on a labeling probability corresponding to the actual methylation status of each target dinucleotide site and the first prediction probability; and
performing iterative parameter update processing on the second prediction model based on the second loss function to obtain the third prediction model, the third prediction model being configured to determine a methylation status of a dinucleotide site in a whole genome sequence.

6. The method according to claim 5, wherein the determining, based on the second feature vector of each second sample whole genome sequence, the first prediction probability that the target dinucleotide site comprised in each second sample whole genome sequence is in the methylated state comprises:
performing, for each second sample whole genome sequence:
performing multi-level feature conversion processing on the second feature vector to obtain a converted second feature vector;
calling an attention mechanism to perform aggregation processing based on the converted second feature vector to obtain a global feature; and
performing activation processing based on the global feature of the second sample whole genome sequence to obtain the first prediction probability that the target dinucleotide site is in the methylated state.

7. The method according to claim 5, wherein the determining the second loss function of the second prediction model based on the labeling probability corresponding to the actual methylation status of each target dinucleotide site and the first prediction probability comprises:
determining, for each target dinucleotide site, a binary cross-entropy loss between the labeling probability corresponding to the actual methylation status and the first prediction probability; and
taking a mean of a plurality of binary cross-entropy losses as the second loss function of the second prediction model.

8. The method according to claim 1, wherein before the pre-training the first prediction model based on the plurality of first sample whole genome sequences to obtain the second prediction model, the method further comprises:
performing whole genome sequencing processing on a biological sample to obtain a plurality of real whole genome sequences; and
taking a preconfigured reference whole genome sequence and the plurality of real whole genome sequences as the first sample whole genome sequences.

9. The method according to claim 8, wherein the performing whole genome sequencing processing on the biological sample to obtain the plurality of real whole genome sequences comprises:
fragmenting deoxyribonucleic acid, and preparing a fragmentation result into text data for sequencing;
sequencing the text data to obtain a plurality of fragment sequencing reads;
determining an arrangement order of bases in a genome sequence based on the plurality of fragment sequencing reads; and
reconstructing the genome sequence based on the arrangement order to obtain the plurality of real whole genome sequences.

10. The method according to claim 1, further comprising:
performing bisulfite treatment on each first sample whole genome sequence to obtain a plurality of treated first sample whole genome sequences, each of the treated first sample whole genome sequences comprising a plurality of treated dinucleotide sites; and
determining an actual methylation status of each dinucleotide site based on dinucleotide sites before treatment and a corresponding plurality of treated dinucleotide sites.

11. The method according to claim 10, wherein the determining the actual methylation status of each dinucleotide site based on dinucleotide sites before treatment and the corresponding plurality of treated dinucleotide sites comprises:
aligning, for each first sample whole genome sequence, the dinucleotide sites before treatment with the plurality of treated dinucleotide sites to obtain a plurality of alignment results corresponding to each dinucleotide site;
determining that the methylation status of the dinucleotide site is an unmethylated state when the alignment result indicates that cytosine of the dinucleotide site is converted to uracil, or is not converted;
determining that the methylation status of the dinucleotide site is a methylated state when the alignment result indicates that cytosine of the dinucleotide site is converted to guanine; and
determining, for each dinucleotide site, the actual methylation status of the dinucleotide site based on a second ratio of a methylated count of the dinucleotide site to a total occurrence count of the dinucleotide site, the second ratio being a probability of methylation of the dinucleotide site.

12. The method according to claim 1, wherein after the performing training processing on the second prediction model based on each annotated second sample whole genome sequence to obtain the trained third prediction model, the method further comprises:
acquiring a third training dataset, the third training dataset comprising a plurality of third whole genome sequences and sample label probabilities of sample dinucleotide sites respectively corresponding to the third whole genome sequences;
calling the third prediction model to perform prediction processing based on the plurality of third whole genome sequences to obtain a second prediction probability that a sample dinucleotide site in each third whole genome sequence is in a methylated state;
determining a first difference between the sample label probability and the second prediction probability, and determining a first number of the first differences that are less than a preconfigured difference; and
taking, in response to an accuracy of the third prediction model being less than a preconfigured accuracy, the third prediction model as the second prediction model, and proceeding to the operation of performing training processing on the second prediction model based on each annotated second sample whole genome sequence to obtain a trained third prediction model, the accuracy being a ratio of the first number to a total number of the sample dinucleotide sites.

13. The method according to claim 1, wherein after the performing training processing on the second prediction model based on each annotated second sample whole genome sequence to obtain a trained third prediction model, the method further comprises:
calling the third prediction model based on the to-be-detected whole genome sequence to perform:
performing feature extraction processing on the to-be-detected whole genome sequence to obtain a fourth feature vector of the to-be-detected whole genome sequence;
determining a third prediction probability that a to-be-detected dinucleotide site comprised in the second sample whole genome sequence is in a methylated state based on the fourth feature vector; and
determining that the to-be-detected dinucleotide site is in the methylated state in response to the third prediction probability being greater than a preconfigured probability.

14. An electronic device comprising:
a memory storing a plurality of instructions; and
a processor configured to execute the plurality of instructions, wherein upon execution of the plurality of instructions, the processor is configured to:
pre-train a first prediction model based on a plurality of first sample whole genome sequences to obtain a second prediction model;
extract a target dinucleotide site from a plurality of dinucleotide sites of each first sample whole genome sequence, and take a sequence within a preconfigured window range comprising the target dinucleotide site as a second sample whole genome sequence;
annotate an actual methylation status for the target dinucleotide site in each second sample whole genome sequence to obtain an annotated second sample whole genome sequence; and
perform training processing on the second prediction model based on each annotated second sample whole genome sequence to obtain a trained third prediction model, the third prediction model being configured to predict a methylation status of a dinucleotide site in a to-be-detected whole genome sequence.

15. The electronic device according to claim 14, wherein to pre-train the first prediction model based on the plurality of first sample whole genome sequences to obtain the second prediction model, the processor, upon execution of the plurality of instructions, is configured to:
call the first prediction model to perform feature extraction processing on the plurality of first sample whole genome sequences to obtain a first feature vector of each first sample whole genome sequence;
determine a first loss function of the first prediction model based on a plurality of first feature vectors; and
perform iterative parameter update processing on the first prediction model based on the first loss function to obtain the second prediction model, the second prediction model being configured to extract a feature vector of the to-be-detected whole genome sequence.

16. The electronic device according to claim 14, wherein to take the sequence within the preconfigured window range comprising the target dinucleotide site as the second sample whole genome sequence, the processor, upon execution of the plurality of instructions, is configured to:
intercept, with the target dinucleotide site as a reference site, the sequence within the preconfigured window range from the first sample whole genome sequence as the second sample whole genome sequence, the reference site being located at any one of the following positions in the preconfigured window range: a boundary of the preconfigured window range, or an interior of the preconfigured window range.

17. The electronic device according to claim 14, wherein to perform training processing on the second prediction model based on each annotated second sample whole genome sequence to obtain the trained third prediction model, the processor, upon execution of the plurality of instructions, is configured to:
call the second prediction model to:
perform feature extraction processing on each second sample whole genome sequence to obtain a second feature vector of each second sample whole genome sequence;
determine, based on the second feature vector of each second sample whole genome sequence, a first prediction probability that the target dinucleotide site comprised in each second sample whole genome sequence is in a methylated state;
determine a second loss function of the second prediction model based on a labeling probability corresponding to the actual methylation status of each target dinucleotide site and the first prediction probability; and
perform iterative parameter update processing on the second prediction model based on the second loss function to obtain the third prediction model, the third prediction model being configured to determine a methylation status of a dinucleotide site in a whole genome sequence.

18. The electronic device according to claim 14, wherein before the processor pre-trains the first prediction model based on the plurality of first sample whole genome sequences to obtain the second prediction model, the processor, upon execution of the plurality of instructions, is further configured to:
perform whole genome sequencing processing on a biological sample to obtain a plurality of real whole genome sequences; and
take a preconfigured reference whole genome sequence and the plurality of real whole genome sequences as the first sample whole genome sequences.

19. The electronic device according to claim 14, wherein the processor, upon execution of the plurality of instructions, is further configured to:
perform bisulfite treatment on each first sample whole genome sequence to obtain a plurality of treated first sample whole genome sequences, each of the treated first sample whole genome sequences comprising a plurality of treated dinucleotide sites; and
determine an actual methylation status of each dinucleotide site based on dinucleotide sites before treatment and a corresponding plurality of treated dinucleotide sites.

20. A non-transitory computer-readable storage medium storing a plurality of instructions executable by a processor, wherein when executed by the processor, the plurality of instructions is configured to cause the processor to:
pre-train a first prediction model based on a plurality of first sample whole genome sequences to obtain a second prediction model;
extract a target dinucleotide site from a plurality of dinucleotide sites of each first sample whole genome sequence, and take a sequence within a preconfigured window range comprising the target dinucleotide site as a second sample whole genome sequence;
annotate an actual methylation status for the target dinucleotide site in each second sample whole genome sequence to obtain an annotated second sample whole genome sequence; and
perform training processing on the second prediction model based on each annotated second sample whole genome sequence to obtain a trained third prediction model, the third prediction model being configured to predict a methylation status of a dinucleotide site in a to-be-detected whole genome sequence.


SEQUENCE LISTING (5 sequences)

SEQ ID NO: 1 - DNA, 23 bp, synthetic construct
  source 1..23: mol_type = other DNA
aacaacggtagatcaggattaga

SEQ ID NO: 2 - DNA, 19 bp, synthetic construct
  source 1..19: mol_type = other DNA
aacaacggtaggtcaggat

SEQ ID NO: 3 - DNA, 18 bp, synthetic construct
  source 1..18: mol_type = other DNA
cggtaggtcaggattaga

SEQ ID NO: 4 - DNA, 23 bp, synthetic construct
  source 1..23: mol_type = other DNA
aagaagggtaggtcaggattaga

SEQ ID NO: 5 - DNA, 20 bp, synthetic construct
  source 1..20: mol_type = other DNA
aacgttaggtcaggattaga